(19) United States
(12) Patent Application Publication
(10) Pub. No.: US 2026/0258446 A1
(43) Pub. Date: Sep. 3, 2026

(54) SMALL MOLECULE ENHANCERS FOR ADENO-ASSOCIATED VIRUS PRODUCTION

(71) Applicant: Mirus Bio LLC, Madison, WI (US)

(72) Inventors: Laura Juckem, Madison, WI (US); Rebecca Mary Reese, Madison, WI (US); Austin Steven Storck, Madison, WI (US); Jennifer Swanson, Madison, WI (US)

(21) Appl. No.: 19/375,879

(22) PCT Filed: May 1, 2024
(86) PCT No.: PCT/US2024/027265

§ 371 Date: Oct. 31, 2025

Related U.S. Application Data
(60) Provisional application No. 63/499,618, filed on May 2, 2023.

(51) Int. Cl. C12N 15/86

(52) U.S. Cl. CPC C12N 15/86; C12N 2750/14143; C12N 2750/14151

(57) ABSTRACT

Described herein are methods for adeno-associated virus (AAV) production using an enhancer capable of increasing AAV production. Also described herein is the use of such enhancers for increasing the production of AAV with a transfection reagent.

### CROSS-REFERENCE TO RELATED APPLICATIONS

This application claims the benefit of U.S. Provisional Patent Application No. 63/499,618, filed May 2, 2023, which is entirely incorporated herein by reference.

### TECHNICAL FIELD

This disclosure relates to enhancers production of recombinant adeno-associated viruses (AAV). The disclosure also includes methods, systems, and kits for AAV production with a transfection enhancer. Preferred enhancers are small molecules.

### BACKGROUND

Adeno-associated viruses were first described as a contaminant of adenovirus preparations and require the presence of a helper virus like adenoviruses to successfully replicate. Recombinant adeno-associated viruses (AAV) are being used in a number of clinical trials as vectors to deliver gene therapies. AAV has been approved as a vehicle for gene therapy which for systemic indications requires a high number of viral genomes per patient dose. The success of gene therapies has prompted the need for more efficient methods of AAV manufacturing to reduce overall costs including by the use of enhancers. Many existing AAV production enhancers are serotype-specific. Thus, while enhancing production for a particular AAV serotype, they do not provide desired enhancement for other serotypes.

### SUMMARY OF THE INVENTION

The present disclosure provides methods, compositions and systems for the production of recombinant adeno-associated virus (AAV) using an enhancer. The enhancer can improve AAV production and/or stability of the cell lines used in the AAV production.

In an aspect, described herein is a method for adeno-associated virus (AAV) production, the method comprising: transfecting eukaryotic cells in a medium with one or more AAV plasmids using a transfection reagent; and contacting the cells with an enhancer capable of increasing AAV production, wherein the enhancer comprises Compound A, Compound B, Compound C, Compound D, Compound E, Compound F, Compound G, sodium valproate, suberoylanilide hydroxamic acid (SAHA), or vinblastine sulfate, or a combination thereof. Compound A is (R)-4-((8-cyclopentyl-7-ethyl-5-methyl-6-oxo-5,6,7,8-tetrahydropteridin-2-yl)amino)-3-methoxy-N-(1-methylpiperidin-4-yl)benzamide. Compound B is(S)-1,2,3,10-tetramethoxy-7-(methylamino)-6,7-dihydrobenzo[a]heptalen-9 (5H)-one. Compound C is [(1S,2S,3R,4S,7R,9S, 10S, 12R, 15S)-4-acetyloxy-1,9,12-trihydroxy-15-[(2R,3S)-2-hydroxy-3-[(2-methylpropan-2-yl)oxycarbonylamino]-3-phenylpropanoyl]oxy-10,14,17,17-tetramethyl-11-oxo-6-oxatetracyclo[11.3.1.0^{3,10}.0^{4,7}]heptadec-13-en-2-yl]benzoate. Compound D is 3-(4-(4-(2-(3-((dimethylamino)methyl)phenyl)-1H-pyrrolo[2,3-b]pyridin-4-yl)-1-ethyl-1H-pyrazol-3-yl)phenyl)-1,1-dimethylurea. Compound E is 4-(dimethylamino)-N-(7-(hydroxyamino)-7-oxoheptyl)benzamide. Compound F is [(1S,2S,3R,4S,7R,9S,10S,12R, 15S)-4,12-diacetyloxy-15-[(2R,3S)-3-benzamido-2-hydroxy-3-phenylpropanoyl]oxy-1,9-dihydroxy-10,14,17,17-tetramethyl-11-oxo-6-oxatetracyclo[11.3.1.0^{3,10}.0^{4,7}]heptadec-13-en-2-yl]benzoate. Compound G is N-[2-[(1R,8S)-4-[[4-(cyclobutylamino)-5-(trifluoromethyl)pyrimidin-2-yl]amino]-11-azatricyclo[6.2.1.0^{2,7}]undeca-2 (7),3,5-trien-11-yl]-2-oxoethyl]acetamide.

In another aspect, described herein is a method increasing AAV production in a stable producer line, the method comprising: contacting the eukaryotic cells with a small molecule enhancer and one or more essential AAV production components; wherein the one or more essential AAV production components have constitutive and/or conditional expression through alteration of a cellular genome, and wherein the small molecule enhancer is one or more selected from the group consisting of: Compound A, Compound B, Compound C, Compound D, Compound E, Compound F, Compound G, sodium valproate or valproic acid, suberoylanilide hydroxamic acid (SAHA), vinblastine, or vinblastine sulfate, or a salt thereof.

In still another aspect, described herein is a composition comprising: an enhancer capable of increasing adeno-associated virus (AAV) production, wherein the enhancer comprises Compound A, Compound B, Compound C, Compound D, Compound E, Compound F, Compound G, sodium valproate or valproic acid, suberoylanilide hydroxamic acid (SAHA), vinblastine, or vinblastine sulfate, or a combination thereof.

In another aspect, described herein is an adeno-associated virus (AAV) production system comprising: Human Embryonic Kidney (HEK) 293 cells (HEK 293 cells or 293 cells) or a derivative of 293 cells (e.g., adherent HEK 293 cells); a cell culture media that supports growth and expansion of the 293 cells; a transfection reagent; and an enhancer capable of increasing AAV production, wherein the enhancer comprises Compound A, Compound B, Compound C, Compound D, Compound E, Compound F, Compound G, sodium valproate or valproic acid, suberoylanilide hydroxamic acid (SAHA), vinblastine, or vinblastine sulfate, or a combination thereof.

In still another aspect, described herein is a method for producing recombinant adeno-associated virus (AAV), comprising: culturing eukaryotic cells in a medium; transfecting the eukaryotic cells in the medium with an AAV plasmid using a transfection reagent; and adding to the medium one or more enhancers increasing AAV production, wherein the one or more enhancers comprise a first enhancer and a second enhancer, wherein the first enhancer and the second enhancer are synergistic to increase the AAV production. As used herein, the terms “AAV plasmid” or “AAV plasmids” are generally defined as one or more plasmids which encode the essential viral components including, for example, the transfer or cis plasmid (encoding the gene of interest, promoter and inverse terminal repeats (ITRs)), the RepCap or trans plasmid (encoding AAV replication (Rep) and capsid (Cap) protein), and the helper plasmid (encoding adenovirus E4, E2A and VA). Helper plasmids can also functionally be substituted by intact adenovirus.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The following drawings form part of the specification and are included to further demonstrate certain embodiments. In some instances, embodiments can be best understood by referring to the accompanying figures in combination with the detailed description presented herein. The description and accompanying figures may highlight a certain specific example, or a certain embodiment. However, one skilled in the art will understand that portions of the example or embodiment may be used in combination with other examples or embodiments.

FIG. 1 is a schematic of the experimental design to identify small molecules that enhance AAV production.

FIGS. 2A-B shows an assessment of top candidates in enhancing the production of AAV2, AAV5, AAV8, and AAV9 serotypes. FIG. 2A shows the AAV genome titer and FIG. 2B shows the percentage of full AAV capsids.

FIGS. 3A-B shows enhancement of AAV8 production in two different suspension HEK293 cell lines: 293-VP 2.0 cells, and Expi293F™ cells. FIG. 3A shows the AAV genome titer and FIG. 3B shows the percentage of full AAV capsids.

FIGS. 4A-B shows enhancement of AAV production when transfecting cells with TransIT-VirusGen® (Mirus Bio LLC), FectoVIR®-AAV (Polyplus), or PEIPro® (Polyplus). FIG. 4A shows the AAV genome titer and FIG. 4B shows the percentage of full AAV capsids.

FIGS. 5A-B shows enhancement of AAV production when the small molecules were added post-transfection. FIG. 5A shows the AAV genome titer and FIG. 5B shows the percentage of full AAV capsids.

FIGS. 6A-B shows enhancement of AAV production when the small molecules were added prior to transfection. FIG. 6A shows the AAV genome titer and FIG. 6B shows the percentage of full AAV capsids.

The novel features of the disclosure are set forth with particularity in the appended claims. A better understanding of the features and advantages of the present disclosure will be obtained by reference to the following detailed description that sets forth illustrative embodiments.

### DETAILED DESCRIPTION

This disclosure relates to the treatment of cells with small molecules to enhance the production of AAV as measured by viral genomes. These enhancers result in a significant increase of recombinant AAV genomes across many serotypes and 293-derived cell types.

So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

It is to be understood that all terminology used herein is for the purpose of describing particular embodiments only and is not intended to be limiting in any manner or scope. For example, as used in this specification and the appended claims, the singular forms “a,” “an” and “the” can include plural referents unless the content clearly indicates otherwise. Thus, for example, reference to “a cell” includes a single cell, as well as two or more cells. Similarly, the word “or” is intended to include “and” unless the context clearly indicate otherwise. The word “or” means any one member of a particular list and also includes any combination of members of that list. Further, all units, prefixes, and symbols may be denoted in its SI accepted form.

Numeric ranges recited within the specification are inclusive of the numbers defining the range and include each integer within the defined range. Throughout this disclosure, various embodiments of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

The term “about,” as used herein, refers to variation in the numerical quantity that can occur, for example, through typical measuring techniques and equipment, with respect to any quantifiable variable, including, but not limited to, volume, time, viral titer, and concentration. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations. The term “about” also encompasses amounts that differ due to different equilibrium conditions for a composition resulting from a particular initial mixture. Whether or not modified by the term “about,” the claims include equivalents to the quantities.

### Recombinant Adeno-Associated Virus (AAV) Production

The term “cell” as used herein includes all types of eukaryotic. In some embodiments, the term refers to eukaryotic cells, especially mammalian cells. In certain embodiments, the term “cell” is meant to refer to human embryonic kidney (HEK) 293 cells or 293 cells, or a variant thereof, such as, e.g., a 293 variant that can grow in suspension. In some embodiments, it can mean variants of 293 cells that can grow, proliferate and be transfected in suspension culture, in particular those variants that can be cultured at high density (e.g., ≥about 2×10⁶ cells/ml, greater ≥about 3×10⁶ cells/ml, or even optionally ≥about 4×10⁶ cells/ml). An example of a variant are 293F cells, such as EXPI293F™ cells or Viral Production Cells 2.0.

As noted above, there are some AAV production enhancers commercially available. These enhancers may work for some AAV serotypes but may not work for other AAV serotypes. As such, different AAV production enhancers may be needed for different AAV serotypes. This necessitates the costly and complicated use of different enhancers for different serotypes. Similarly, many existing enhancers are only compatible with particular 293 cell variants. Further, there are transfection reagents used as part of the transfection process for AAV production. Many existing enhancers are limited to compatibility with particular transfection reagents, thereby making them inapplicable in certain production processes.

Beneficially, the enhancers described herein not only provide significant improvement in cell production, but also present broad compatibility across AAV serotypes, 293 cell variants, and transfection reagents. In an embodiment, the enhancer is compatible with multiple AAV serotypes, multiple 293 cell variants, and/or multiple transfection reagents.

In some embodiments, the term “high density” or “high-density” when used in the context of culturing cells and conducting transfection workflows, generally refers to a known cell line, or a variant of a known cell line, that can be grown or cultured in an appropriate cell culture medium to densities of ≥about 1×10⁶ cells/ml, ≥about 2×10⁶ cells/ml, ≥about 3×10⁶ cells/mL, ≥about 4×10⁶ cells/mL or even optionally ≥about 5×10⁶ cells/mL, while still retaining the ability to be transfected at high efficiency and are able to express a target AAV vector at high titer, for example 5×10¹⁰ viral genomes per mL (vg/mL) or more in an unpurified cell culture.

In some embodiments, the cells are adapted for high-density cell culture. This refers to a cell lineage or a (non-clonal) population of cells derived from the same parental cell lineage that has been adapted to grow at high density in a high-density culture medium while retaining cell viability at or above about 80%. Such cells may be isolated or selected out from the parental population of cells by maintaining the cells at high density ≥about 20, 30, 40, 50, 60, 70, or 80 sequential passages and gradually replacing the proportion of growth medium with the desired high-density culture medium. Optionally, during the process, different pools of cells may be individually propagated and subjected to the selection procedure while simultaneously assessing transfection efficiency and or AAV vector production efficiency, so that clonal population of cells may be selected that can be sustained and grown at high density, transfected with high efficiency, and express high titers of AAV. Alternatively, cells could be genetically modified to alter cellular characteristics which may augment transfection, viral vector or protein production. Clonal populations of cells may be generated using know methods and techniques, for example, flow cytometry sorting and/or single cell cloning. In certain embodiments, flow cytometry sorting is used to isolate cell clones adapted for high-density cell culture and use in the production of AAV. In some embodiments, cell clones adapted for high-density cell culture and use in the production of AAV are obtained via single cell cloning and confirmed as single cell clones using known techniques, such as imaging. While it may be readily apparent to the skilled practitioner that a variety of cell types and lineages may be subjected to this selection procedure, it has been determined that cell lineages derived from, for example, 293 cells are particularly amenable to the selection process for being adapted to high-density growth conditions. In some embodiments, cells that are adapted to high-density growth culture and amenable for use herein may also be capable of being transfected at high efficiency and/or capable of expressing AAV vector at yield exceeding at least about 5×10⁹ vg/mL, at least about 1×10¹⁰ vg/mL, at least about 5×10¹⁰ vg/mL, at least about 1×10¹¹ vg/mL, at least about 5×10¹¹ vg/mL, at least about 1×10¹² vg/mL, at least about 5×10¹² vg/mL, at least about 1×10¹³ vg/mL, or at least about 5×10¹³ vg/mL of unconcentrated AAV vectors. In some embodiments, cells adapted for high-density culture used are capable of being sustained and transfected at densities in the range from about 1×10⁶ cells/mL to about 2×10⁷ cells/mL, about 1×10⁶ cells/mL to about 3×10⁶ cells/mL, about 2×10⁶ cells/mL to about 4×10⁶ cells/mL, about 2.5×10⁶ cells/mL to about 4×10⁶ cells/mL, or about 3×10⁶ cells/mL to about 5×10⁶ cells/mL. In some embodiments, cells may be adapted for high-density culture and transfected at densities in the range from about 1×10⁶ cells/mL to about 2×10⁷ cells/mL, from about 1×10⁶ cells/mL to about 5×10⁶ cells/mL, from about 1×10⁶ cells/mL to about 4×10⁶ cells/mL, from about 1×10⁶ cells/mL to about 3×10⁶ cells/mL, from about 1×10⁶ cells/mL to about 2×10⁶ cells/mL.

In some embodiments, the cells are grown in a suspension culture. This includes a cell culture in which the majority or all of the cells in a culture vessel are present in suspension, and the minority or none of the cells in the culture vessel are attached to the vessel surface or to another surface within the vessel. In some embodiments, suspension culture has ≥about 75% of the cells in the culture vessel are in suspension, not attached to a surface on or in the culture vessel. In some embodiments, a suspension culture has ≥about 85% of the cells in the culture vessel are present in suspension, not attached to a surface on or in the culture vessel. In some embodiments, suspension culture has ≥about 95% of the cells in the culture vessel present in suspension, not attached to a surface on or in the culture vessel.

A variety of cell culture media may be used to culture the cells. The phrases “cell culture medium”, “tissue culture medium”, “culture medium” (plural “media” in each case) and “medium formulation” refer to a nutritive solution for cultivating cells or tissues. These phrases can be used interchangeably.

Serum free media are often desired by investigators. Any medium, including serum free medium, that supports the growth of the cells described herein may be used. The medium may also be protein free. A “serum-free medium” is a medium that contains no serum (e.g., fetal bovine serum (FBS), calf serum, horse serum, goat serum, human serum, etc.) and is generally designated by the letters SFM. The phrase “protein-free” culture media refers to culture media that contain no protein (e.g., no serum proteins such as serum albumin or attachment factors, nutritive proteins such as growth factors, or metal ion carrier proteins such as transferrin, ceruloplasmin, etc.). In some embodiments, if peptides are present, the peptides are smaller peptides, e.g., di- or tri-peptides. In some embodiments, peptides of deca-peptide length or greater are no more than about 1%, no more than about 0.1%, and no more than about 0.01% of the amino acids present in the protein free medium.

In some embodiments, a high-density culture media may be used, including any culture medium capable of sustaining the growth of mammalian cells. In some embodiments, cells are grown in suspension at densities of up to about 2×10⁷ cells/mL while maintaining cell viability in excess of about 80%, such as more than about 90%, and further, maintaining the ability of said suspension cells to be efficiently transfected with one or more AAV plasmids and express high amounts of AAV vector. The high-density culture medium used may vary between different applications and uses, and may depend on the nature of the cell line being used, the nature of the transfection modality selected for transfer of the expression vector into cells, and the amount and nature of any production enhancers added to the system as described herein. In certain embodiments, high-density culture medium used in the present systems and methods is serum-free and protein-free. In certain embodiments, the cell culture medium allows the cultivation and growth of suspension cells to a density of up to about 2×10⁷ cells/mL, for example up to about 1.2×10⁷ cells/mL, or between about 2×10⁶ cells/mL to about 1×10⁷ cells/mL. In certain embodiments, the culture medium used may enable the viral titer produced in the transient expression system to exceed at least 1×10¹⁰ vg/mL up to about 1×10¹² vg/mL, or up to about 2×10¹¹ vg/mL. In some embodiments, the high-density culture medium used may facilitate the transfection of cells at densities in the range of about 1×10⁶ to about 20×10⁶ cells/mL, about 1×10⁶ to about 4×10⁶ cells/mL, or about 2.5×10⁶ to about 3×10⁶ cells/mL.

Examples of culture media suitable for use herein include, though are not limited to, HuMEC Basal Serum free Medium, KNOCKOUT™ CTS™ XenoFREE ESC/iPSC Medium, STEMPRO™-34 SFM Medium, STEMPRO™ NSC Medium, ESSENTIAL™-8 Medium, Medium 254, Medium, 106, Medium, 131, Medium, 154, Medium, 171, Medium 171, Medium 200, Medium 231, HeptoZYME-SFM, Human Endothelial-SFM, GIBCO® FREESTYLE™ 293 Expression Medium, Medium 154CF/PRF, Medium 154C, Medium 154 CF, Medium 106, Medium 200PRF, Medium 131, Essential™-6 Medium, STEMPRO™-34 Medium, Gibco® Astrocyte Medium, AIM VR Medium CTS™, AMINOMAX™ C-100 Basal Medium, AMINOMAX™-II Complete Medium, CD FORTICHO™ Medium, CD CHO AGT Medium, CHO—S—SFM Medium, GIBCO® FREESTYLE™ CHO Expression Medium, CD OPTICHO™ Medium, CD CHO Medium, CD DG44 Medium, SF-900™ Medium, EXPI293™ Expression Medium, LHC Basal Medium, LHC-8 Medium, 293 SFM Medium, CD 293 Medium, AEM Growth Medium, PER.C6® Cell Medium, AIM VR Medium, EXPILIFE® Medium, Keratinocyte-SFM Medium, LHC Medium, LHC-8 Medium, LHC-9 Medium, and any derivatives or modifications thereof. In certain embodiments, a culture media may be CD FORTICHO™ Medium, CD CHO AGT Medium, CHO—S—SFM Medium, GIBCO® FREESTYLE™ CHO Expression Medium, CD OPTICHO™ Medium, CD CHO Medium, CD DG44 Medium, GIBCO® FREESTYLE™ 293 Expression Medium, EXPI293™ Expression Medium, LV-MAX™ Production Medium, Viral Production Medium, FREESTYLE™ F17 Expression Medium, DYNAMIS™ Medium, BALANCD® HEK293 medium, or a like medium, or a modified version thereof. The culture media may be any media that is suitable (e.g., formulated) for the high-density growth, propagation, transfection and maintenance of 293 cells, a 293 cell variant, or any other cells adapted for use in a high-density culture system.

The AAV production system can also comprise a transfection reagent or a composition that facilitates entry of a macromolecule into a cell. In certain embodiments, the transfection reagent comprises a cationic lipid, an amphipathic compound, a cationic acrylamide copolymer, a cationic cyclic amine, or combination thereof; examples of these compounds are disclosed in U.S. Pat. Nos. 8,921,448, 9,290,779, 9,677,077, 9,856,496, and 10,619,162, the detailed disclosure and figures of each is incorporated herein by reference in its entirety.

In some embodiments, a reagent for the introduction of macromolecules into cells can comprise one or more lipids which can be cationic lipids and/or neutral lipids. Suitable lipids include, but are not limited to, N-[1-(2,3-dioleyloxy) propyl]-N,N,N-trimethylamonium chloride (DOTMA), dioleoylphosphatidylcholine (DOPE), 1,2-Bis(oleoyloxy)-3-(4′-trimethylammonio) propane (DOTAP), dihydroxyl-dimyristylspermine tetrahydrochloride (DHDMS), hydroxyl-dimyristylspermine tetrahydrochloride (HDMS), 1,2-dioleoyl-3-(4′-trimethylammonio) butanoyl-sn-glycerol (DOTB), 1,2-dioleoyl-3-succinyl-sn-glycerol choline ester (DOSC), cholesteryl (4′-trimethylammonio) butanoate (ChoTB), cetyltrimethylammonium bromide (CTAB), 1,2-dioleoyl-3-dimethyl-hydroxyethyl ammonium bromide (DORI), 1,2-dioleyloxypropyl-3-dimethyl-hydroxyethyl ammonium bromide (DOME), 1,2-dimyristyloxypropyl-3-dimethylhydroxyethyl ammonium bromide (DMRIE), O,O′-didodecyl-N-[p(2-trimethylammonioethyloxy)benzoyl]-N,N,N-trimethylammonium chloride, spermine conjugated to one or more lipids (for example, 5-carboxyspermylglycine dioctadecylamide (DOGS), N,N^{I},N^{II},N^{III}-tetramethyl-N,N^{I},N^{II},N^{III}-tet-rapalmitylspermine (TM-TPS) and dipalmitoylphasphatidylethanolamine 5-carboxyspermylaminde (DPPES)), lipopolylysine (polylysine conjugated to DOPE), TRIS (Tris(hydroxymethyl)aminomethane, tromethamine) conjugated fatty acids (TFAs) and/or peptides such as trilysyl-alanyl-TRIS mono-, di-, and tri-palmitate, (3B—[N—(N′,N′-dimethylaminoethane)-carbamoyl]cholesterol (DCChol), N-(α-trimethylammonioacetyl)-didodecyl-D-glutamate chloride (TMAG), dimethyl dioctadecylammonium bromide (DDAB), 2,3-dioleyloxy-N-[2 (spermine-carboxamido)ethyl]-N,N-dimethyl-1-propanamin-iniumtrinuoroacetate (DOSPA) and combinations thereof.

In some embodiments, the transfection reagent may further comprise at least one additional helper lipid. Helper lipids are known in the art and include, but are not limited to, neutral lipids. In certain embodiments, the neutral lipid is selected from the group consisting of DOPE, DOPC and cholesterol. In certain embodiments, the transfection reagent comprises at least one cationic lipid and at least one neutral lipid.

Those skilled in the art may appreciate that certain combinations of the above mentioned lipids have been shown to be particularly suited for the introduction of nucleic acids into cells including, for example, a 3:1 (w/w) combination of DOSPA and DOPE is available from Life Technologies Corporation, Carlsbad, Calif. under the trade name LIPOFECTAMINE™, a 1:1 (w/w) combination of DOTMA and DOPE is available from Thermo Fisher Scientific under the trade name LIPOFECTIN®, a 1:1 (M/M) combination of DIVIRIE and cholesterol is available from Life Technologies Corporation, Carlsbad, Calif. under the trade name DMRIE-C reagent; and a 1:1.5 (M/M) combination of TM-TPS and DOPE is available from Life Tech. In some embodiments, the transfection reagent is a cationic lipid transfection reagent. In some embodiments, the transfection reagent is a polymer-based transfection reagent. Other commercially available cationic lipid transfection reagents include, without limitation, TRANSFAST™ (available from Promega Corporation); LYOVEC™ (available from InvivoGen); DOTAP liposomal transfection reagent (available from Roche); TRANSIT® transfection reagents (available from Mirus); and Insect GENEJUICE® Transfection Reagent (EMD Millipore). Additional transfection reagents that may be used herein include, without limitation, LIPOFECTAMINE® 2000, LIPOFECTAMINE® 3000, available from Thermo Fisher Scientific; VIAFECT™ Transfection Reagent, FUGENE® 6 Transfection Reagent, and FUGENE® HD Transfection Reagent, each of which is available from Promega Corporation; and TRANSFECTIN™ Lipid Reagent, available from BioRad Laboratories, Inc.

In certain embodiments, the transfection reagent comprises a cationic polymer. Cationic polymers represent the second major class of transfection reagents. Suitable cationic polymers include, but are not limited to, a linear or branched polyethyleneimine (PEI) and variants thereof, PEI dendrimers, a polypropyleneimine (PPI), poly(amidoamine) (PAA) and dendrimers (PAMAM), cationic cyclodextrin, polyalkylamine, a polyhydroxyalkylamine, poly(butyleneimine) (PBI), spermine, a N-substituted polyallylamine, N-substituted chitosan, a N-substituted polyornithine, a N-substituted polylysine (PLL), a N-substituted polyvinylamine, poly(β-amino ester), hyperbranched poly(amino ester) (h-PAE), networked poly(amino ester) (n-PAE), poly(4-hydroxy-1-proline ester) (PHP-ester) and a poly-β-aminoacid. In certain embodiments, the transfection reagent is FectoVIR®-AAV Transfection Reagent (VWR) or PEIpro® DNA Transfection Reagent for Virus Production (VWR).

In certain embodiments, the transfection reagent is combined with the AAV transfer vector to form a DNA/transfection reagent complex prior to addition to the cells. In certain embodiments, the transfection reagent is combined with the AAV transfer vector, the pRep/Cap plasmid and the pHelper plasmid to form a DNA/transfection reagent complex prior to addition to the cells.

An embodiment disclosed herein is an AAV production system. The AAV production system comprises an AAV production enhancer. It can further comprise one or more of 293 cells or a derivative of 293 cells, a cell culture media that supports growth of 293 cells, and a transfection reagent.

In certain embodiments, the AAV production enhancer comprises a histone deacetylase (HDAC) inhibitor, a kinase inhibitor, an anti-mitotic agent, or a combination thereof. In certain embodiments, the HDAC inhibitor comprises sodium valproate, Compound E (M344), suberoylanilide hydroxamic acid (SAHA), or a combination thereof. In certain embodiments, the kinase inhibitor comprises Compound A, Compound D, Compound G, or a combination thereof. In certain embodiments, the anti-mitotic agent comprises Compound B (colcemid), Compound C (docetaxel), Compound F (paclitaxel), or vinblastine sulfate, or a combination thereof.

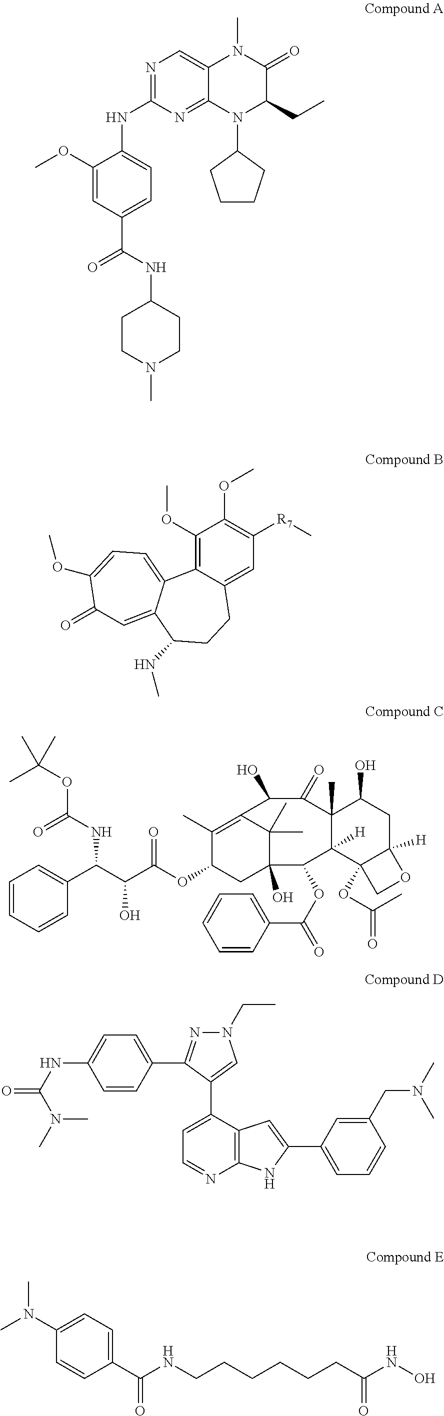

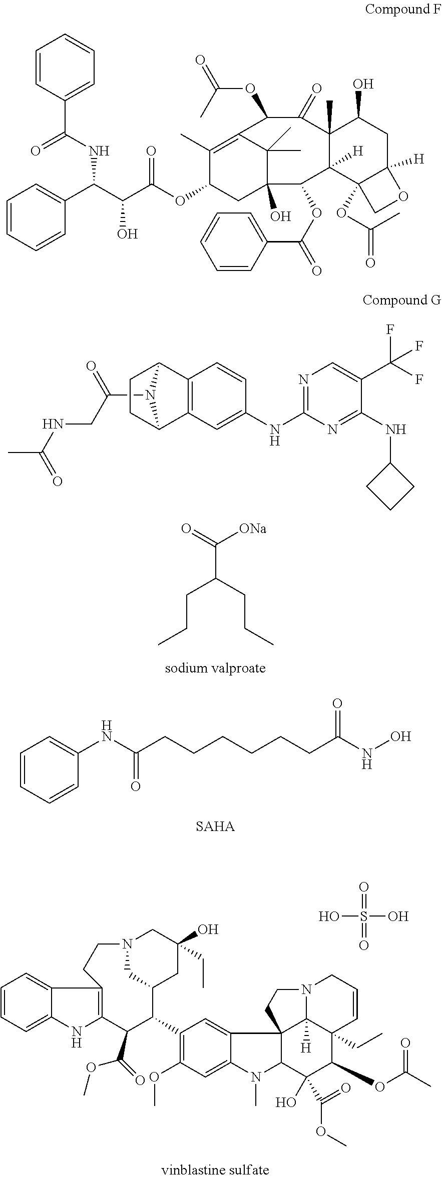

In certain embodiments, Compound A is included at a concentration (final concentration contacted with the cells) from about 0.01 μM to about 5 μM, from about 0.01 μM to about 4 μM, from about 0.01 μM to about 3 μM, from about 0.01 μM to about 2 μM, from about 0.01 μM to about 1 μM, from about 0.01 μM to about 0.75 μM, from about 0.025 μM to about 5 μM, from about 0.025 μM to about 4 μM, from about 0.025 μM to about 3 μM, from about 0.025 μM to about 2 μM, from about 0.025 μM to about 1 μM, or from about 0.025 μM to about 0.75 μM. In certain embodiments, Compound A is included at a concentration from about 0.05 μM to about 0.15 M. The concentration may be any value or subrange within the recited ranges, including endpoints.

In certain embodiments, Compound B is included at a concentration (final concentration contacted with the cells) from about 0.01 μM to about 5 μM, from about 0.01 μM to about 2 μM, from about 0.01 μM to about 1 μM, from about 0.01 μM to about 0.75 μM, from about 0.01 μM to about 0.5 μM, from about 0.01 μM to about 0.25 μM, from about 0.05 μM to about 5 μM, from about 0.05 μM to about 2 μM, from about 0.05 μM to about 1 μM, from about 0.05 μM to about 0.75 μM, from about 0.05 μM to about 0.5 M, or from about 0.05 μM to about 0.25 μM. In some embodiments, Compound B is included at a concentration from about 0.05 μM to about 0.20 μM, or from about 0.06 μM to about 0.20 μM. The concentration may be any value or subrange within the recited ranges, including endpoints.

In certain embodiments, Compound C is included at a concentration (final concentration contacted with the cells) from about 0.1 μM to about 50 μM, from about 0.1 μM to about 40 M, from about 0.1 μM to about 30 μM, from about 0.1 μM to about 20 μM, from about 0.1 μM to about 10 μM, from about 0.1 μM to about 5 μM, from about 1 μM to about 50 μM, from about 1 μM to about 40 μM, from about 1 μM to about 30 μM, from about 1 μM to about 20 μM, from about 1 μM to about 10 μM, or from about 1 μM to about 5 μM. In certain embodiments, Compound C is included at a concentration from about 1 μM to about 20 μM. The concentration may be any value or subrange within the recited ranges, including endpoints.

In certain embodiments, Compound D is included at a concentration (final concentration contacted with the cells) from about 0.01 μM to about 5 μM, from about 0.01 μM to about 4 μM, from about 0.01 μM to about 3 μM, from about 0.01 μM to about 2 μM, from about 0.01 μM to about 1 μM, from about 0.01 μM to about 0.75 μM, from about 0.025 μM to about 5 μM, from about 0.025 μM to about 4 μM, from about 0.025 μM to about 3 μM, from about 0.025 μM to about 2 μM, from about 0.025 μM to about 1 μM, or from about 0.025 μM to about 0.75 μM. In certain embodiments, Compound D is included at a concentration from about 0.01 μM to about 0.25 μM, or from about 0.25 μM to about 1 μM. The concentration may be any value or subrange within the recited ranges, including endpoints.

In certain embodiments, Compound E is included at a concentration (final concentration contacted with the cells) from about 0.01 μM to about 10 μM, from about 0.01 μM to about 5 μM, from about 0.01 μM to about 4 μM, from about 0.01 μM to about 3 μM, from about 0.01 μM to about 2 μM, from about 0.01 μM to about 1 μM, from about 0.1 μM to about 10 μM, from about 0.1 μM to about 5 μM, from about 0.1 μM to about 4 μM, from about 0.1 μM to about 3 μM, from about 0.1 μM to about 2.5 μM, from about 0.1 μM to about 2 μM, from about 0.1 μM to about 1 μM, from about 0.5 μM to about 10 μM, from about 0.5 μM to about 5 μM, from about 0.5 μM to about 4 μM, from about 0.5 μM to about 3 μM, from about 0.5 μM to about 2 μM, or from about 0.5 μM to about 1 μM. In some embodiments, Compound E is included at a concentration from about 0.5 μM-1.0 μM. The concentration may be any value or subrange within the recited ranges, including endpoints.

In certain embodiments, Compound F is included at a concentration (final concentration contacted with the cells) from about 0.05 μM to about 100 μM, from about 0.05 μM to about 50 μM, from about 0.05 μM to about 40 μM, from about 0.05 μM to about 30 μM, from about 0.05 μM to about 20 μM, from about 0.05 μM to about 10 μM, from about 0.05 μM to about 5 μM, from about 0.5 μM to about 100 μM, from about 0.5 μM to about 50 μM, from about 0.5 μM to about 40 μM, from about 0.5 μM to about 30 μM, from about 0.5 μM to about 20 μM, from about 0.5 μM to about 10 μM, from about 0.5 μM to about 5 μM. The concentration may be any value or subrange within the recited ranges, including endpoints.

In certain embodiments, Compound G is included at a concentration (final concentration contacted with the cells) from about 0.001 μM to about 1 μM, from about 0.001 μM to about 0.5 μM, from about 0.001 μM to about 0.1 μM, from about 0.001 μM to about 0.05 μM, from about 0.005 μM to about 1 μM, from about 0.005 μM to about 0.5 μM, from about 0.005 UM to about 0.1 μM, from about 0.005 μM to about 0.05 μM. In certain embodiments, Compound G is included at a concentration of about 0.01 μM. The concentration may be any value or subrange within the recited ranges, including endpoints.

In certain embodiments, sodium valproate is included at a concentration (final concentration contacted with the cells) from about 100 μM to about 3000 μM, from about 100 μM to about 2500 μM, from about 100 μM to about 2000 μM, from about 100 μM to about 1500 μM, from about 100 μM to about 1000 μM, from about 100 μM to about 900 μM, from about 100 μM to about 800 μM, from about 100 μM to about 700 μM, from about 100 μM to about 600 μM, from about 100 μM to about 500 μM, from about 400 μM to about 3000 μM, from about 400 μM to about 2500 μM, from about 400 μM to about 2000 μM, from about 400 μM to about 1500 μM, from about 400 μM to about 1000 μM, from about 400 μM to about 900 μM, from about 400 μM to about 800 μM, from about 400 μM to about 700 μM, from about 400 μM to about 600 μM, or from about 400 μM to about 500 μM. The concentration may be any value or subrange within the recited ranges, including endpoints.

In certain embodiments, vinblastine sulfate is included at a concentration (final concentration contacted with the cells) from about 1 μM to about 30 μM, from about 1 μM to about 15 μM, from about 1 μM to about 10 μM, from about 1 μM to about 5 μM, from about 2.5 μM to about 30 μM, from about 2.5 μM to about 15 μM, from about 2.5 μM to about 10 μM, from about 2.5 M to about 5 μM. The concentration may be any value or subrange within the recited ranges, including endpoints.

In certain embodiments, SAHA is included at a concentration (final concentration contacted with the cells) from about 0.01 μM to about 10 μM, from about 0.01 μM to about 5 μM, from about 0.01 μM to about 4 μM, from about 0.01 μM to about 3 μM, from about 0.01 μM to about 2 μM, from about 0.01 μM to about 1 μM, from about 0.5 μM to about 10 μM, from about 0.5 μM to about 5 μM, from about 0.5 μM to about 4 μM, from about 0.5 μM to about 3 μM, from about 0.5 μM to about 2 μM, from about 0.5 μM to about 1 μM. The concentration may be any value or subrange within the recited ranges, including endpoints.

In certain embodiments, two or more enhancers are used. In certain embodiments, the two or more enhancers are Compound A and Compound D. In certain embodiments, Compound A is from about 0.025 μM to about 1 μM, and Compound D is from about 0.025 μM to about 1 μM. In certain embodiments, Compound A is about 0.025 μM, and Compound D is about 0.025 μM, about 0.05 μM, about 0.1 μM, about 0.2 μM, about 0.4 μM, about 0.6 μM, about 0.8 μM, or about 1.0 μM. In certain embodiments, Compound A is about 0.05 μM, and Compound D is about 0.025 μM, about 0.05 μM, about 0.1 μM, about 0.2 μM, about 0.4 μM, about 0.6 μM, about 0.8 μM, or about 1.0 μM. In certain embodiments, Compound A is about 0.1 μM, and Compound D is about 0.025 μM, about 0.05μ, about 0.1μ, about 0.2μ, about 0.4μ, about 0.6μ, about 0.8μ, or about 1.0μ. In certain embodiments, Compound A is about 0.2 μM, and Compound D is about 0.025 μM, about 0.05 μM, about 0.1 μM, about 0.2 μM, about 0.4 μM, about 0.6 μM, about 0.8 μM, or about 1.0 μM. In certain embodiments, Compound A is about 0.4 μM, and Compound D is about 0.025 μM, about 0.05μ, about 0.1μ, about 0.2μ, about 0.4μ, about 0.6μ, about 0.8μ, or about 1.0μ. In certain embodiments, Compound A is about 0.6 μM, and Compound D is about 0.025 μM, about 0.05 μM, about 0.1 μM, about 0.2 μM, about 0.4 μM, about 0.6 μM, about 0.8 μM, or about 1.0 μM. In certain embodiments, Compound A is about 0.8 μM, and Compound D is about 0.025 μM, about 0.05 μM, about 0.1 μM, about 0.2 μM, about 0.4 μM, about 0.6 μM, about 0.8 μM, or about 1.0 μM. In certain embodiments, Compound A is about 1.0 μM, and Compound D is about 0.025 μM, about 0.05 μM, about 0.1 μM, about 0.2 μM, about 0.4 μM, about 0.6 μM, about 0.8 μM, or about 1.0 μM.

In certain embodiments, the two or more enhancers are Compound C and Valproic Acid Sodium Salt. In certain embodiments, Compound C is from about 1.0 μM to about 20 μM, and Valproic Acid Sodium Salt is from about 400 μM to about 600 μM. In certain embodiments, Compound C is about 1.0 μM, and Valproic Acid Sodium Salt is about 400 μM, about 425 μM, about 450 μM, about 500 μM, about 525 μM, about 550 μM, about 575 μM, or about 600 μM. In certain embodiments, Compound C is about 2.0 μM, and Valproic Acid Sodium Salt is about 400 μM, about 425 μM, about 450 μM, about 500 μM, about 525 μM, about 550 μM, about 575 μM, or about 600 μM. In certain embodiments, Compound C is about 4.0 μM, and Valproic Acid Sodium Salt is about 400 μM, about 425 μM, about 450 μM, about 500 μM, about 525 μM, about 550 μM, about 575 μM, or about 600 μM. In certain embodiments, Compound C is about 6.0 μM, and Valproic Acid Sodium Salt is about 400 μM, about 425 μM, about 450 μM, about 500 μM, about 525 μM, about 550 μM, about 575 μM, or about 600 μM. In certain embodiments, Compound C is about 8.0 μM, and Valproic Acid Sodium Salt is about 400 M, about 425 μM, about 450 μM, about 500 μM, about 525 μM, about 550 μM, about 575 μM, or about 600 μM. In certain embodiments, Compound C is about 10 μM, and Valproic Acid Sodium Salt is about 400 μM, about 425 μM, about 450 μM, about 500 μM, about 525 μM, about 550 μM, about 575 μM, or about 600 μM. In certain embodiments, Compound C is about 15 μM, and Valproic Acid Sodium Salt is about 400 μM, about 425 μM, about 450 μM, about 500 μM, about 525 μM, about 550 μM, about 575 μM, or about 600 μM. In certain embodiments, Compound C is about 20 μM, and Valproic Acid Sodium Salt is about 400 μM, about 425 μM, about 450 μM, about 500 μM, about 525 μM, about 550 μM, about 575 μM, or about 600 μM.

In certain embodiments, the two or more enhancers are Compound A and Compound G. In certain embodiments, Compound A is from about 0.01 μM to about 0.25 μM, and Compound G is from about 0.02 μM to about 0.5 μM. In certain embodiments, Compound A is about 0.01 μM, and Compound G is about 0.02 μM, about 0.04 μM, about 0.06 μM, about 0.08 UM, about 0.10 μM, about 0.15 μM, about 0.20 μM, or about 0.25 μM. In certain embodiments, Compound A is about 0.02 μM, and Compound G is about 0.02 μM, about 0.04 μM, about 0.06 μM, about 0.08 UM, about 0.10 UM, about 0.15 μM, about 0.20 μM, or about 0.25 μM. In certain embodiments, Compound A is about 0.04 μM, and Compound G is about 0.02 μM, about 0.04 μM, about 0.06 μM, about 0.08 UM, about 0.10 μM, about 0.15 μM, about 0.20 μM, or about 0.25 μM. In certain embodiments, Compound A is about 0.06 μM, and Compound G is about 0.02 μM, about 0.04 μM, about 0.06 μM, about 0.08 UM, about 0.10 μM, about 0.15 μM, about 0.20 μM, or about 0.25 μM. In certain embodiments, Compound A is about 0.08 μM, and Compound G is about 0.02 μM, about 0.04 UM, about 0.06 μM, about 0.08 μM, about 0.10 μM, about 0.15 μM, about 0.20 μM, or about 0.25 μM. In certain embodiments, Compound A is about 0.1 μM, and Compound G is about 0.02 μM, about 0.04 μM, about 0.06 μM, about 0.08 μM, about 0.10 μM, about 0.15 μM, about 0.20 μM, or about 0.25 μM. In certain embodiments, Compound A is about 0.15 μM, and Compound G is about 0.02 μM, about 0.04 μM, about 0.06 μM, about 0.08 UM, about 0.10 μM, about 0.15 μM, about 0.20 μM, or about 0.25 μM.

In certain embodiments, Compound A is about 0.20 μM, and Compound G is about 0.02 μM, about 0.04 μM, about 0.06 μM, about 0.08 UM, about 0.10 μM, about 0.15 μM, about 0.20 μM, or about 0.25 μM. In certain embodiments, Compound A is about 0.25 μM, and Compound G is about 0.02 μM, about 0.04 μM, about 0.06 μM, about 0.08 μM, about 0.10 μM, about 0.15 μM, about 0.20 μM, or about 0.25 μM.

In certain embodiments, the two or more enhancers are Compound D and Compound E. In certain embodiments, Compound D is from about 0.01 μM to about 0.25 μM, and Compound E is from about 0.1 μM to about 2.5 μM. In certain embodiments, Compound D is about 0.01 μM, and Compound E is about 0.1 μM, about 0.2 μM, about 0.4 μM, about 0.8 μM, about 1.0 μM, about 1.5 μM, about 2.0 M, or about 2.5 μM. In certain embodiments, Compound D is about 0.02 μM, and Compound E is about 0.1 μM, about 0.2 μM, about 0.4 μM, about 0.8 μM, about 1.0 μM, about 1.5 μM, about 2.0 μM, or about 2.5 μM. In certain embodiments, Compound D is about 0.04 μM, and Compound E is about 0.1 μM, about 0.2 μM, about 0.4 μM, about 0.8 μM, about 1.0 μM, about 1.5 μM, about 2.0 μM, or about 2.5 μM. In certain embodiments, Compound D is about 0.06 μM, and Compound E is about 0.1 μM, about 0.2 μM, about 0.4 μM, about 0.8 μM, about 1.0 μM, about 1.5 μM, about 2.0 μM, or about 2.5 μM. In certain embodiments, Compound D is about 0.08 UM, and Compound E is about 0.1 μM, about 0.2 μM, about 0.4 μM, about 0.8 μM, about 1.0 μM, about 1.5 μM, about 2.0 μM, or about 2.5 μM. In certain embodiments, Compound D is about 0.1 μM, and Compound E is about 0.1 μM, about 0.2 μM, about 0.4 μM, about 0.8 μM, about 1.0 μM, about 1.5 μM, about 2.0 μM, or about 2.5 μM. In certain embodiments, Compound D is about 0.15 μM, and Compound E is about 0.1 μM, about 0.2 μM, about 0.4 μM, about 0.8 μM, about 1.0 μM, about 1.5 μM, about 2.0 μM, or about 2.5 μM. In certain embodiments, Compound D is about 0.20 μM, and Compound E is about 0.1 μM, about 0.2 μM, about 0.4 μM, about 0.8 μM, about 1.0 μM, about 1.5 μM, about 2.0 μM, or about 2.5 μM. In certain embodiments, Compound D is about 0.25 μM, and Compound E is about 0.1 μM, about 0.2 μM, about 0.4 μM, about 0.8 μM, about 1.0 μM, about 1.5 μM, about 2.0 μM, or about 2.5 μM.

In certain embodiments, the AAV production enhancer is added at one or more than one time point, such as at the time of transfection (about hour 0) until about 24 hours after transfection. In some embodiments, the enhancer may be added prior to the time of transfection. In some embodiments, the enhancer may be added at the time of transfection. In some embodiments, the enhancer may be added up to about 4 hours after transfection. In some embodiments, the AAV production enhancer may be added about 5 seconds, 10 seconds, 30 seconds, 1 minute, 2 minutes, 3 minutes, 4 minutes, 5 minutes, 10 minutes, 15 minutes, 30 minutes, 45 minutes, 1 hour, 2 hours, 3 hours, or 4 hours prior to transfection. In some embodiments, AAV production enhancer may be added from about 5 seconds, 10 seconds, 30 seconds, 1 minute, 2 minutes, 3 minutes, 4 minutes, 5 minutes, 10 minutes, 15 minutes, 30 minutes, 45 minutes, 1 hour, 2 hours, 3 hours, 4 hours, 5 hours, 6 hours, 7 hours, 8 hours, 9 hours, 10 hours, 11 hours, 12 hours, 16 hours, 24 hours after transfection, or later. In some embodiments, the AAV production enhancer may be added from about 1 hour prior to transfection to about 4 hours after transfection. The AAV production enhancer may be added at any time (or subrange) within the recited ranges, including endpoints.

The design and production of AAV vectors is known in the art. See, e.g., U.S. Pat. Nos. 5,354,678; 6,759,237; 5,753,500; and 5,474,935. For proper packaging of AAV, packaging plasmids can be used. These plasmids encode genes that are necessary for packaging of AAV vectors. Such genes include genes expressing the capsid protein (cap) and the replication (rep) gene. Alternatively, the genes may be stably expressed by the cells. The AAV genes can be any from any serotype AAV, including but not limited to serotype 1, 2, 3, 4, 5, 6, 7, 8, 9, DJ or DJ/8. Packaging plasmids encoding AAV rep and cap genes are often referred to as pAAV-RC, pRep/Cap or pRC plasmids. AAV transfer vectors, packaging plasmids, packaging cell lines, and other products for AAV production are commercially available, for example from Agilent, Aldevron, Genemedi, Cell Biolabs, Inc., Vector Biolabs, Addgene, Clontech, and Thermo Fisher Scientific.

In certain embodiments, helper virus (e.g., from adenovirus or herpesvirus) components are required for proper function of the AAV production system. Helper virus components may be present on plasmids (and often referred to as pAAV-Helper or pHelper plasmid) or otherwise present in the cells. Helper virus components include, but are not limited to, E1A, E1B, E2A, E4, and/or VA.

Preferably the enhancer comprises a carrier. In an embodiment, the enhancer can be in a powder form, which is resuspended in a carrier. Preferred carriers are solvents safe for use with the enhancer and overall AAV production system. Preferred solvents are organic solvents. Preferred solvents include, but are not limited to, polar protic solvents, polar aprotic solvents, and polar diprotic solvents. Preferred solvents, include, but are not limited to dimethyl sulfoxide (DMSO), ethanol, water, mixtures thereof.

As used herein, the terms “enhancer”, “production enhancer”, “AAV and production enhancer” are used interchangeably. For the avoidance of double, the “enhancer” is not a nucleic acid, a peptide, or part of an AAV vector.

In some embodiments, a lowered amount of the one or two enhancer contributes to the improved compatibility with two or more AAV serotypes, two or more 293 cell variants, and/or two or more transfection reagents used in the AAV production. For example, when an enhancer is used above a threshold level in the cell culture, the enhancer is less compatible with at least one AAV serotype, at least one 293 cell variant, and/or at least one transfection reagent in AAV production compared with when the enhancer is used below the threshold level in otherwise the same cell culture. In some embodiments, the AAV serotype includes but is not limited to serotype 1, 2, 3, 4, 5, 6, 7, 8, 9, DJ or DJ/8.

In some embodiments, when a first enhancer and a second enhancer are used, the first enhancer and/or the second enhancer can be used at a higher concentration than when either the first enhancer or the second enhancer is used individually, thereby allowing higher efficacy of AAV production when the combination of enhancers is used.

In some embodiments, when a first enhancer and a second enhancer are used, the combination of the first enhancer and the second enhancer disclosed herein have a synergistic effect to the utilization of the first enhancer or the second enhancer alone. In some embodiments, a synergistic effect of two or more enhancers is an effect the combination of the two or more enhancers, which effect is greater than the additive effects of the two or more enhancers. In some embodiments; a synergistic effect of a combination of two or more enhancers permits the use of lower amounts of the first enhancer or the second enhancer and/or less frequent administration of the first enhancer or the second enhancer to the medium/cell culture. In some embodiments, the ability to utilize lower amount of the first enhancer or the second enhancer and/or to administer the first enhancer or the second enhancer less frequently reduces the undesired effects associated with the administration of the first enhancer or the second enhancer to the medium/cell culture without reducing the efficacy of the first enhancer or the second enhancer in the AAV production. In some embodiments, a synergistic effect results in improved efficacy of the first enhancer or the second enhancer in producing the AAV particles. In some embodiments, the efficacy is the AAV particles measured in vg/mL in the cell culture. In some embodiments, a synergistic effect results in improved compatibility with two or more AAV serotypes, two or more 293 cell variants, and/or two or more transfection reagents used in the AAV production. For example, when individually administered, a first enhancer is compatible with a first AAV serotype but not a second AAV serotype while a second enhancer is compatible with the second AAV serotype but not the first AAV serotype. In contrast, the combination of the first enhancer and the second enhancer is compatible with both the first AAV serotype and the second AAV serotype. In some embodiments, the AAV serotype includes but is not limited to serotype 1, 2, 3, 4, 5, 6, 7, 8, 9, DJ or DJ/8.

### Methods for Adeno Associated Virus (AAV) Production

An AAV production system as described herein can be used to produce AAV vectors. Methods for AAV production comprising culturing eukaryotic cells in a medium; transfecting the cells with one or more AAV plasmids using a transfection reagent; and contacting the cells with the enhancer are provided. In certain embodiments, the methods comprise culturing the transfected cells for a period of time sufficient for expression of the AAV, and harvesting the AAV. In certain embodiments, the cells are cultured in suspension culture.

An AAV production enhancer as described herein can be used to increase production of AAV vectors. Methods for increasing production of adeno-associated virus (AAV) comprising contacting AAV-transfected eukaryotic cells with the enhancer are provided.

In certain embodiments, the enhancer increases viral genome titer of the culture at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2.0-fold, at least about 2.1-fold, at least about 2.2-fold, at least about 2.3-fold, at least about 2.4-fold, at least about 2.5-fold, at least about 2.6-fold, at least about 2.7-fold, at least about 2.8-fold, at least about 2.9-fold, at least about 3.0-fold, at least about 3.1-fold, at least about 3.2-fold, at least about 3.3-fold, at least about 3.4-fold, at least about 3.5-fold, at least about 3.6-fold, at least about 3.7-fold, at least about 3.8-fold, at least about 3.9-fold, at least about 4.0-fold, at least about 4.1-fold, at least about 4.2-fold, at least about 4.3-fold, at least about 4.4-fold, at least about 4.5-fold, at least about 4.6-fold, at least about 4.7-fold, at least about 4.8-fold, at least about 4.9-fold, at least about 5.0-fold, at least about 5.1-fold, at least about 5.2-fold, at least about 5.3-fold, at least about 5.4-fold, at least about 5.5-fold, at least about 5.6-fold, at least about 5.7-fold, at least about 5.8-fold, at least about 5.9-fold, at least about 6.0-fold, at least about 6.1-fold, at least about 6.2-fold, at least about 6.3-fold, at least about 6.4-fold, at least about 6.5-fold, at least about 6.6-fold, at least about 6.7-fold, at least about 6.8-fold, at least about 6.9-fold, or at least about 7.0-fold.

In certain embodiments, the method includes titering the harvested AAV. The AAV may be titered using any method. In certain embodiments, the AAV is titered using polymerase chain reaction (PCR). In certain embodiments, the AAV is titered using quantitative PCR (qPCR). In certain embodiments, the AAV is titered using digital droplet PCR (ddPCR). In certain embodiments, the AAV is titered using digital PCR (dPCR). In certain embodiments, the AAV is titered using ELISA. In certain embodiments, the AAV is titered using a viral titer kit; see also, U.S. Pat. No. 6,841,357, which is incorporated herein by reference in its entirety. In certain embodiments, the AAV is titered by determining the concentration of viral particles that can transduce cells (infectious titer), e.g. by cell transduction assay. In certain embodiments, the AAV is titered using DNA dot blotting.

In certain embodiments, the harvested AAV has a titer of at least about 1×10¹⁰ viral genomes per milliliter (vg/mL). In certain embodiments, the harvested AAV has a titer of at least about 2×10¹⁰ vg/mL. In certain embodiments, the harvested AAV has a titer of at least about 3×10¹⁰ vg/mL. In certain embodiments, the harvested AAV has a titer of at least about 4×10¹⁰ vg/mL. In certain embodiments, the harvested AAV has a titer of at least about 5×10¹⁰ vg/mL. In certain embodiments, the harvested AAV has a titer of at least about 6×10¹⁰ vg/mL. In certain embodiments, the harvested AAV has a titer of at least about 7×10¹⁰ vg/mL. In certain embodiments, the harvested AAV has a titer of at least about 8×10¹⁰ vg/mL. In certain embodiments, the harvested AAV has a titer of at least about 9×10¹⁰ vg/mL. In certain embodiments, the harvested AAV has a titer of at least about 1×10¹¹ vg/mL. In certain embodiments, the harvested AAV has a titer of at least about 2×10¹¹ vg/mL. In certain embodiments, the harvested AAV has a titer of at least about 3×10¹¹ vg/mL. In certain embodiments, the harvested AAV has a titer of at least about 4×10¹¹ vg/mL. In certain embodiments, the harvested AAV has a titer of at least about 5×10¹¹ vg/mL. In certain embodiments, the harvested AAV has a titer of at least about 6×10¹¹ vg/mL. In certain embodiments, the harvested AAV has a titer of at least about 7×10¹¹ vg/mL. In certain embodiments, the harvested AAV has a titer of at least about 8×10¹¹ vg/mL. In certain embodiments, the harvested AAV has a titer of at least about 9×10¹¹ vg/mL.

In certain embodiments, the harvested AAV has a titer between about 1×10¹⁰ vg/mL and about 1×10¹² vg/mL. In certain embodiments, the harvested AAV has a titer between about 2×10¹⁰ vg/mL and about 1×10¹² vg/mL. In certain embodiments, the harvested AAV has a titer between about 3×10¹⁰ vg/mL and about 1×10¹² vg/mL. In certain embodiments, the harvested AAV has a titer between about 4×10¹⁰ vg/mL and about 1×10¹² vg/mL. In certain embodiments, the harvested AAV has a titer between about 5×10¹⁰ vg/mL and about 1×10¹² vg/mL. In certain embodiments, the harvested AAV has a titer between about 6×10¹⁰ vg/mL and about 1×10¹² vg/mL. In certain embodiments, the harvested AAV has a titer between about 7×10¹⁰ vg/mL and about 1×10¹² vg/mL. In certain embodiments, the harvested AAV has a titer between about 8×10¹⁰ vg/mL and about 1×10¹² vg/mL. In certain embodiments, the harvested AAV has a titer between about 9×10¹⁰ vg/mL and about 1×10¹² vg/mL. In certain embodiments, the harvested AAV has a titer between about 1×10¹¹ vg/mL and about 1×10¹² vg/mL. In certain embodiments, the harvested AAV has a titer between about 2×10¹¹ vg/mL and about 1×10¹² vg/mL. In certain embodiments, the harvested AAV has a titer between about 3×10¹¹ vg/mL and about 1×10¹² vg/mL. In certain embodiments, the harvested AAV has a titer between about 4×10¹¹ vg/mL and about 1×10¹² vg/mL. In certain embodiments, the harvested AAV has a titer between about 5×10¹¹ vg/mL and about 1×10¹² vg/mL. In certain embodiments, the harvested AAV has a titer between about 2×10¹⁰ vg/mL and about 9×10¹¹ vg/mL. In certain embodiments, the harvested AAV has a titer between about 2×10¹⁰ vg/mL and about 8×10¹¹ vg/mL. In certain embodiments, the harvested AAV has a titer between about 2×10¹⁰ vg/mL and about 7×10¹¹ vg/mL. In certain embodiments, the harvested AAV has a titer between about 2×10¹⁰ vg/mL and about 6×10¹¹ vg/mL. In certain embodiments, the harvested AAV has a titer between about 2×10¹⁰ vg/mL and about 5×10¹¹ vg/mL. In certain embodiments, the harvested AAV has a titer between about 2×10¹⁰ vg/mL and about 4×10¹¹ vg/mL. In certain embodiments, the harvested AAV has a titer between about 2×10¹⁰ vg/mL and about 3×10¹¹ vg/mL. In certain embodiments, the harvested AAV has a titer between about 2×10¹⁰ vg/mL and about 2×10¹¹ vg/mL. In certain embodiments, the harvested AAV has a titer between about 2×10¹⁰ vg/mL and about 1×10¹¹ vg/mL. The titer may be any value or subrange within the recited ranges, including endpoints.

In certain embodiments, the enhancer increases the percentage of full capsids at least about 1.5-fold, at least about 1.6-fold, at least about 1.7-fold, at least about 1.8-fold, at least about 1.9-fold, at least about 2.0-fold, at least about 2.1-fold, at least about 2.2-fold, at least about 2.3-fold, at least about 2.4-fold, at least about 2.5-fold, at least about 2.6-fold, at least about 2.7-fold, at least about 2.8-fold, at least about 2.9-fold, at least about 3.0-fold, at least about 3.1-fold, at least about 3.2-fold, at least about 3.3-fold, at least about 3.4-fold, at least about 3.5-fold, at least about 3.6-fold, at least about 3.7-fold, at least about 3.8-fold, at least about 3.9-fold, at least about 4.0-fold, at least about 4.1-fold, at least about 4.2-fold, at least about 4.3-fold, at least about 4.4-fold, at least about 4.5-fold, at least about 4.6-fold, at least about 4.7-fold, at least about 4.8-fold, at least about 4.9-fold, at least about 5.0-fold, at least about 5.1-fold, at least about 5.2-fold, at least about 5.3-fold, at least about 5.4-fold, at least about 5.5-fold, at least about 5.6-fold, at least about 5.7-fold, at least about 5.8-fold, at least about 5.9-fold, at least about 6.0-fold, at least about 6.1-fold, at least about 6.2-fold, at least about 6.3-fold, at least about 6.4-fold, at least about 6.5-fold, at least about 6.6-fold, at least about 6.7-fold, at least about 6.8-fold, at least about 6.9-fold, or at least about 7.0-fold.

In certain embodiments, the method includes quantitation of capsids. In certain embodiments, capsids are quantified using ELISA. In certain embodiments, capsids are quantified using a homogeneous based luciferase assay utilizing capsid specific antibodies. The percentage of full capsids can be calculated by dividing the total number of genomes by the total number of capsids. Other suitable detection methods for full capsids include, but are not limited to, anion exchange chromatography (AEX), Stunner (from Unchained Labs, a combination ultraviolet-visible (UV-Vis) spectrophotometer, static light scattering (SLS), and DLS instrument designed to rapidly assess genomic and viral capsid titers, empty/full ratio, etc.), and charge detection mass spectrometry (CDMS).

As used herein, the percentage of full capsids is calculated from the ratio of detected full capsids over the detected, crude AAV (pre-purification). In certain embodiments, the percentage of full capsids is at least about 5%. In certain embodiments, the percentage of full capsids is at least about 6%. In certain embodiments, the percentage of full capsids is at least about 7%. In certain embodiments, the percentage of full capsids is at least about 8%. In certain embodiments, the percentage of full capsids is at least about 9%. In certain embodiments, the percentage of full capsids is at least about 10%. In certain embodiments, the percentage of full capsids is at least about 11%. In certain embodiments, the percentage of full capsids is at least about 12%. In certain embodiments, the percentage of full capsids is at least about 13%. In certain embodiments, the percentage of full capsids is at least about 14%. In certain embodiments, the percentage of full capsids is at least about 15%. In certain embodiments, the percentage of full capsids is at least about 20%. In certain embodiments, the percentage of full capsids is at least about 25%. In certain embodiments, the percentage of full capsids is at least about 30%. In certain embodiments, the percentage of full capsids is at least about 35%. In certain embodiments, the percentage of full capsids is at least about 40%. In certain embodiments, the percentage of full capsids is at least about 45%. In certain embodiments, the percentage of full capsids is at least about 50%.

In certain embodiments, the percentage of full capsids is from about 5% to about 50%. In certain embodiments, the percentage of full capsids is from about 5% to about 40%. In certain embodiments, the percentage of full capsids is from about 5% to about 30%. In certain embodiments, the percentage of full capsids is from about 5% to about 20%. In certain embodiments, the percentage of full capsids is from about 5% to about 10%. In certain embodiments, the percentage of full capsids is from about 10% to about 50%. In certain embodiments, the percentage of full capsids is from about 10% to about 40%. In certain embodiments, the percentage of full capsids is from about 10% to about 30%. In certain embodiments, the percentage of full capsids is from about 10% to about 20%. In certain embodiments, the percentage of full capsids is from about 15% to about 50%. In certain embodiments, the percentage of full capsids is from about 15% to about 40%. In certain embodiments, the percentage of full capsids is from about 15% to about 30%. In certain embodiments, the percentage of full capsids is from about 15% to about 20%. In certain embodiments, the percentage of full capsids is from about 20% to about 50%. In certain embodiments, the percentage of full capsids is from about 20% to about 40%. In certain embodiments, the percentage of full capsids is from about 20% to about 30%. In certain embodiments, the percentage of full capsids is from about 15% to about 20%. The percentage of full capsids may be any value or subrange within the recited ranges, including endpoints.

The cells can be cultured in any suitable culture vessel. By “culture vessel” is meant any container, for example, a glass, plastic, or metal container, that can provide an aseptic environment for culturing cells. In certain embodiments, the cells are cultured in a bioreactor. In certain embodiments, the cells are cultured in a flask.

The cells may be cultured in any volume of cell culture medium that supports growth of the cells and production of AAV. In certain embodiments, the cells are cultured in a volume of about 1 milliliter (mL) to about 5000 liters (L). In certain embodiments, the cells are cultured in a volume of about 2 mL to about 5000 L. In certain embodiments, the cells are cultured in a volume of about 15 mL to about 5000 L. In certain embodiments, the cells are cultured in a volume of about 30 mL to about 5000 L. In certain embodiments, the cells are cultured in a volume of about 50 mL to about 5000 L. In certain embodiments, the cells are cultured in a volume of about 100 mL to about 5000 L. In certain embodiments, the cells are cultured in a volume of about 500 mL to about 5000 L. In certain embodiments, the cells are cultured in a volume of about 1 L to about 5000 L. In certain embodiments, the cells are cultured in a volume of about 10 L to about 5000 L. In certain embodiments, the cells are cultured in a volume of about 1 mL to about 200 L. In certain embodiments, the cells are cultured in a volume of about 2 mL to about 200 L. In certain embodiments, the cells are cultured in a volume of about 15 mL to about 200 L. In certain embodiments, the cells are cultured in a volume of about 30 mL to about 200 L. In certain embodiments, the cells are cultured in a volume of about 50 mL to about 200 L. In certain embodiments, the cells are cultured in a volume of about 100 mL to about 200 L. In certain embodiments, the cells are cultured in a volume of about 500 mL to about 200 L. In certain embodiments, the cells are cultured in a volume of about 1 L to about 200 L. In certain embodiments, the cells are cultured in a volume of about 10 L to about 200 L. In certain embodiments, the cells are cultured in a volume of about 2 mL to about 100 L. In certain embodiments, the cells are cultured in a volume of about 2 mL to about 50 L. In certain embodiments, the cells are cultured in a volume of about 2 mL to about 20 L. In certain embodiments, the cells are cultured in a volume of about 2 mL to about 5 L. In certain embodiments, the cells are cultured in a volume of about 2 mL to about 1 L. In certain embodiments, the cells are cultured in a volume of about 2 mL to about 500 mL. In certain embodiments, the cells are cultured in a volume of about 500 mL to about 10 L. In certain embodiments, the cells are cultured in a volume of about 1 L to about 10 L. The culture volume may be any value or subrange within the recited ranges, including endpoints.

### Method for Using the Enhancers to Stabilize Producer Cell Lines

The enhancers as described herein can be cell stability enhancers that can be used to increase the stability of the producer cell lines, including but not limited to, 293 cells and multiple 293 cell variants. Methods for increasing the stability of the producer cell lines (with or without the integrated AAV vector) comprise contacting the producer cell lines (with or without the integrated AAV vector) with the enhancer(s) herein described.

In some embodiments, methods for using the small molecule enhancer to augment AAV production in a producer cell line (PCL) comprise: culturing eukaryotic cells in a medium; and contacting the cells with the small molecule enhancer. In some embodiments, the methods comprise culturing the eukaryotic in suspension culture together with the small molecule enhancer. In some embodiments, the methods comprise culturing stably and/or transiently transfected eukaryotic cells with the small molecule enhancer.

In certain embodiments, the small molecule enhancer comprises a histone deacetylase (HDAC) inhibitor, a kinase inhibitor, an anti-mitotic agent, or a combination thereof. In certain embodiments, the HDAC inhibitor comprises sodium valproate, Compound E (M344), suberoylanilide hydroxamic acid (SAHA), or a combination thereof. In certain embodiments, the kinase inhibitor comprises Compound A, Compound D, Compound G, or a combination thereof (structures of which are shown below). In certain embodiments, the anti-mitotic agent comprises Compound B (colcemid), Compound C (docetaxel), Compound F (paclitaxel), vinblastine sulfate, or a combination thereof.

### EMBODIMENTS

The following numbered embodiments also form part of the present disclosure:
1. A method for increasing production of adeno-associated virus (AAV), the method comprising: contacting AAV-transfected eukaryotic cells with an enhancer capable of increasing AAV production, wherein the enhancer comprises Compound A, Compound B, Compound C, Compound D, Compound E, Compound F, Compound G, sodium valproate, suberoylanilide hydroxamic acid (SAHA), vinblastine sulfate, or a combination thereof.
2. The method of embodiment 1, wherein the enhancer comprises two or more of Compound A, Compound B, Compound C, Compound D, Compound E, Compound F, Compound G, sodium valproate, suberoylanilide hydroxamic acid (SAHA), and vinblastine sulfate.
3. The method of embodiment 1 or embodiment 2, wherein the enhancer comprises Compound A and Compound D.
4. The method of any one of embodiments 1-3, wherein Compound A is at a concentration from about 0.025 μM to about 1 μM, and wherein Compound D is at a concentration from about 0.15 μM to about 1 μM.
5. The method of any one of embodiments 1-4, wherein the enhancer comprises Compound C.
6. The method of any one of embodiments 1-5, wherein Compound C is at a concentration from about 1 μM to about 20 μM.
7. The method of any one of embodiments 1-6, wherein the enhancer comprises Compound C and sodium valproate.
8. The method of any one of embodiments 1-7, wherein Compound C is at a
concentration from about 1 μM to about 20 μM, and wherein sodium valproate is provided at a concentration from about 400 μM to about 600 μM.
9. The method of any one of embodiments 1-8, wherein the enhancer comprises Compound A and Compound G.
10. The method of any one of embodiments 1-9, wherein Compound A is at a concentration from about 0.01 μM to about 0.25 μM, and wherein Compound Gis at a concentration from about 0.02 μM to about 0.5 μM.
11. The method of any one of embodiments 1-10, wherein the enhancer comprises Compound F.
12. The method of any one of embodiments 1-11, wherein Compound F is at a concentration from about 0.5 μM to about 40 μM.
13. The method of any one of embodiments 1-12, wherein the enhancer comprises Compound D.
14. The method of any one of embodiments 1-13, wherein Compound D is at a concentration from about 0.025 μM to about 2.5 μM.
15. The method of any one of embodiments 1-14, wherein the enhancer comprises Compound E and Compound D.
16. The method of any one of embodiments 1-15, wherein Compound E is at a concentration from about 0.1 μM to about 2.5 μM, and wherein Compound D is at a concentration from about 0.01 μM to about 0.25 μM.
17. The method of any one of embodiments 1-16, wherein the enhancer comprises vinblastine sulfate.
18. The method of any one of embodiments 1-17, wherein vinblastine sulfate is at a concentration from about 2.5 μM to about 15 μM.
19. The method of any one of embodiments 1-18, wherein the enhancer comprises SAHA.
20. The method of any one of embodiments 1-19, wherein SAHA is at a concentration from about 0.5 μM to about 1 μM.
21. The method of any one of embodiments 1-20, wherein the enhancer comprises Compound B.
22. The method of any one of embodiments 1-21, wherein Compound B is at a concentration from about 0.05 μM to about 0.25 μM.
23. The method of any one of embodiments 1-22, wherein the cells are 293 cells or a derivative of 293 cells.
24. The method of any one of embodiments 1-23, wherein the enhancer is contacted with the cells within about 4 hours of transfection.
25. The method of any one of embodiments 1-24, further comprising culturing the cells for a period of time sufficient for expression of the AAV; and harvesting the AAV.
26. The method of any one of embodiments 1-25, wherein the enhancer increases viral genome titer of the culture and/or the percentage of full capsids at least 1.5-fold, at least 2-fold, at least 3-fold, at least 4-fold, at least 5-fold, or at least 6-fold.
27. A method for adeno-associated virus (AAV) production, the method comprising: transfecting eukaryotic cells in a medium with one or more AAV plasmids using a transfection reagent; and contacting the eukaryotic cells with an enhancer capable of increasing AAV production, wherein the enhancer comprises Compound A, Compound B, Compound C, Compound D, Compound E, Compound F, Compound G, sodium valproate, suberoylanilide hydroxamic acid (SAHA), vinblastine sulfate, or a combination thereof.
28. The method of embodiment 27, wherein the enhancer comprises two or more of Compound A, Compound B, Compound C, Compound D, Compound E, Compound F, Compound G, sodium valproate, suberoylanilide hydroxamic acid (SAHA), and vinblastine sulfate.
29. The method of embodiment 27 or embodiment 28, wherein the enhancer comprises Compound A and Compound D.
30. The method of any one of embodiments 27-29, wherein Compound A is at a concentration from about 0.025 μM to about 1 μM, and wherein Compound D is at a concentration of about 0.025 μM to about 1 μM.
31. The method of any one of embodiments 27-30, wherein the enhancer comprises Compound C.
32. The method of any one of embodiments 27-31, wherein Compound C is at a concentration from about 1 μM to about 20 μM.
33. The method of any one of embodiments 27-32, wherein the enhancer comprises Compound C and sodium valproate.
34. The method of any one of embodiments 27-33, wherein Compound C is at a concentration from about 1 μM to about 20 μM, and wherein sodium valproate is provided at a concentration from about 400 μM to about 600 μM.
35. The method of any one of embodiments 27-34, wherein the enhancer comprises Compound A and Compound G.
36. The method of any one of embodiments 27-35, wherein Compound A is at a concentration of about from about 0.01 μM to about 0.25 μM, and wherein Compound G is at a concentration from about 0.02 μM to about 0.5 μM.
37. The method of any one of embodiments 27-36, wherein the enhancer comprises Compound F.
38. The method of any one of embodiments 27-37, wherein Compound F is at a concentration from about 0.5 μM to about 40 μM.
39. The method of any one of embodiments 27-38, wherein the enhancer comprises Compound D.
40. The method of any one of embodiments 27-39, wherein Compound D is at a concentration of about 0.05 μM.
41. The method of any one of embodiments 27-40, wherein the enhancer comprises Compound E and Compound D.
42. The method of any one of embodiments 27-41, wherein Compound E is at a concentration from about 0.1 μM to about 2.5 μM, and wherein Compound D is at a concentration from about 0.01 μM to about 0.25 μM.
43. The method of any one of embodiments 27-42, wherein the enhancer comprises vinblastine sulfate.
44. The method of any one of embodiments 27-43, wherein vinblastine sulfate is at a concentration from about 2.5 μM to about 15 μM.
45. The method of any one of embodiments 27-44, wherein the enhancer comprises SAHA.
46. The method of any one of embodiments 27-45, wherein SAHA is at a concentration from about 0.5 μM to about 1 μM.
47. The method of any one of embodiments 27-46, wherein the enhancer comprises Compound B.
48. The method of any one of embodiments 27-47, wherein Compound B is at a concentration from about 0.05 μM to about 0.25 μM.
49. The method of any one of embodiments 27-48, wherein the cells are 293 cells or a derivative of 293 cells.
50. The method of any one of embodiments 27-49, wherein the transfection reagent comprises a cationic lipid, a helper lipid, a cationic polymer, or a combination thereof.
51. The method of any one of embodiments 27-50, wherein the enhancer is provided prior to, during, or after transfection.
52. The method of any one of embodiments 27-51, wherein the enhancer is provided within about 4 hours of transfection.
53. The method of any one of embodiments 27-52, wherein the enhancer increases viral genome titer of the culture and/or the percentage of full capsids at least 1.5-fold, at least 2-fold, at least 3-fold, at least 4-fold, at least 5-fold, or at least 6-fold.
54. The method of any one of embodiments 27-53, further comprising culturing the cells for a period of time sufficient for expression of the AAV; and harvesting the AAV.
55. A composition comprising: an enhancer capable of increasing adeno-associated virus (AAV) production, wherein the enhancer comprises Compound A, Compound B, Compound C, Compound D, Compound E, Compound F, Compound G, sodium valproate, suberoylanilide hydroxamic acid (SAHA), vinblastine sulfate, or a combination thereof.
56. The composition of embodiment 55, wherein the enhancer comprises two or more of Compound A, Compound B, Compound C, Compound D, Compound E, Compound F, Compound G, sodium valproate, suberoylanilide hydroxamic acid (SAHA), and vinblastine sulfate.
57. The composition of embodiment 55 or embodiment 56, wherein the enhancer comprises Compound A and Compound D.
58. The composition of any one of embodiments 55-57, wherein the enhancer comprises Compound C.
59. The composition of any one of embodiments 55-58, wherein the enhancer comprises Compound C and sodium valproate.
60. The composition of any one of embodiments 55-59, wherein the enhancer comprises Compound A and Compound G.
61. The composition of any one of embodiments 55-60, wherein the enhancer comprises Compound F.
62. The composition of any one of embodiments 55-61, wherein the enhancer comprises Compound D.
63. The composition of any one of embodiments 55-62, wherein the enhancer comprises Compound E and Compound D.
64. The composition of any one of embodiments 55-63, wherein the enhancer comprises vinblastine sulfate.
65. The composition of any one of embodiments 55-64, wherein the enhancer comprises SAHA.
66. The composition of any one of embodiments 55-65, wherein the enhancer comprises Compound B.
67. The composition of any one of embodiments 55-66, further comprising a cell culture media that supports growth and expansion of 293 cells.
68. The composition of any one of embodiments 55-67, further comprising a carrier.
69. An adeno-associated virus (AAV) production system comprising: 293 cells or a derivative of 293 cells; a cell culture media that supports growth and expansion of the 293 cells; a transfection reagent; and an enhancer capable of increasing AAV production, wherein the enhancer comprises Compound A, Compound B, Compound C, Compound D, Compound E, Compound F, Compound G, sodium valproate, suberoylanilide hydroxamic acid (SAHA), vinblastine sulfate, or a combination thereof.
70. The AAV production system of embodiment 69, wherein the enhancer comprises two or more of Compound A, Compound B, Compound C, Compound D, Compound E, Compound F, Compound G, sodium valproate, suberoylanilide hydroxamic acid (SAHA), and vinblastine sulfate.
71. The AAV production system of embodiment 69 or embodiment 70, wherein the enhancer comprises Compound A and Compound D.
72. The AAV production system of any one of embodiments 69-71, wherein the enhancer comprises Compound C.
73. The AAV production system of any one of embodiments 69-72, wherein the enhancer comprises Compound C and sodium valproate.
74. The AAV production system of any one of embodiments 69-73, wherein the enhancer comprises Compound A and Compound G.
75. The AAV production system of any one of embodiments 69-74, wherein the enhancer comprises Compound F.
76. The AAV production system of any one of embodiments 69-75, wherein the enhancer comprises Compound D.
77. The AAV production system of any one of embodiments 69-76, wherein the enhancer comprises Compound E and Compound D.
78. The AAV production system of any one of embodiments 69-77, wherein the enhancer comprises vinblastine sulfate.
79. The AAV production system of any one of embodiments 69-78, wherein the enhancer comprises SAHA.
80. The AAV production system of any one of embodiments 69-79, wherein the enhancer comprises Compound B.
81. The AAV production system of any one of embodiments 69-80, wherein the transfection reagent comprises a cationic lipid, a helper lipid, a cationic polymer, or a combination thereof.
82. The AAV production system of any one of embodiments 69-81, further comprising a packaging plasmid.
83. A method for producing recombinant AAV (AAV), comprising: culturing eukaryotic cells in a medium; transfecting the eukaryotic cells in the medium with one or more AAV plasmids using a transfection reagent; and adding to the medium one or more enhancers increasing AAV production, wherein the one or more enhancers comprise a first enhancer at a final concentration of no more than 1 μM in the medium.
84. The method of embodiment 83, wherein the first enhancer is Compound A, Compound B, Compound D, Compound E, Compound F, Compound G, or suberoylanilide hydroxamic acid (SAHA), or a salt thereof.
85. The method of embodiment 83 or embodiment 84, wherein the one or more enhancers further comprise a second enhancer that is different from the first enhancer.
86. The method of embodiment 85, wherein the second enhancer is Compound A, Compound B, Compound C, Compound D, Compound E, Compound F, Compound G, sodium valproate, suberoylanilide hydroxamic acid (SAHA), vinblastine or vinblastine sulfate, or a salt thereof.
87. A method for producing recombinant adeno-associated virus (AAV), comprising: culturing eukaryotic cells in a medium; transfecting the eukaryotic cells in the medium with one or more AAV plasmids using a transfection reagent; and adding to the medium one or more enhancers increasing AAV production, wherein the one or more enhancers comprise a first enhancer and a second enhancer, wherein the first enhancer and the second enhancer are synergistic to increase the AAV production.
88. The method of embodiment 87, wherein each of the one or more enhancers is independently Compound A, Compound B, Compound C, Compound D, Compound E, Compound F, Compound G, sodium valproate, suberoylanilide hydroxamic acid (SAHA), vinblastine or vinblastine sulfate, or a salt thereof.
89. The method of any one of embodiments 83-88, wherein the one or more enhancers are added to the medium starting from no later than five minutes after completing adding the transfection reagent.
90. The method of embodiment 83 or embodiment 87, wherein each of the one or more enhancers is independently an epigenetic modifier, a P13K inhibitor, a cell cycle modulator, an innate immune activator/inhibitor, a proteosome inhibitor, a kinase inhibitor, anti-mitotic agent or microtubule disassembly inhibitor, a histone deacetylase (HDAC) inhibitor, a free fatty acid, an endoplasmic reticulum stress inducer/inhibitor, or an autophagy activator/inhibitor.
91. The method of embodiment 90, wherein the one or more enhancers comprise a kinase inhibitor.
92. The method of embodiment 90 or embodiment 91, wherein the one or more enhancers comprise an anti-mitotic agent or microtubule disassembly inhibitor.
93. The method of any one of embodiments 90-92, wherein the one or more enhancers comprise an HDAC inhibitor.
94. The method of any one of embodiments 83-93, wherein the eukaryotic cells are 293 cells or a derivative of 293 cells.
95. The method of any one of embodiments 83-94, wherein the one or more enhancers are added in one or more batches to the medium.
96. The method of any one of embodiments 83-94, wherein the one or more enhancers are added continuously to the medium within about 4 hours of completing adding the transfection reagent.
97. The method of any one of embodiments 83-96, wherein the first enhancer and the second enhancer are added together.
98. The method of any one of embodiments 83-96, wherein the first enhancer is added before the second enhancer.
99. The method of embodiment 98, wherein the second enhancer is added within 4 hours of adding the first enhancer.
100. The method of any one of embodiments 83-96, wherein the second enhancer is added before the first enhancer.
101. The method of embodiment 100, wherein the first enhancer is added within 4 hours of adding the second enhancer.
102. The method of any one of embodiments 83-96, wherein the first enhancer and/or the second enhancer are added before the transfection reagent is added.
103. The method of embodiment 102, wherein adding the one or more enhancers is complete within about 4 hours of completing adding the transfection reagent.
104. The method of any one of embodiments 83-103, wherein each of the one or more enhancer is not a nucleic acid or a peptide.
105. The method of any one of embodiments 83-104, further comprising: culturing transfected eukaryotic cells for a period time sufficient for expression of the AAV; and harvesting the AAV.
106. The method of embodiment 105, wherein AAV particles harvested in embodiment 102 is at least 1.5-fold, at least 2.0-fold, at least 3.0-fold, at least 4-fold, at least 5-fold, or at least 6-fold as many AAV particles harvested than a culture in absence of adding the one or more enhancers.
107. The method of any one of embodiments 83-106, wherein the transfection reagent comprises a cationic lipid, a helper lipid, a cationic polymer, or a combination thereof.
108. The method of any one of embodiments 83-107, wherein the one or more enhancers are compatible with two or more AAV serotypes, two or more 293 cell variants, and/or two or more transfection reagents.
109. A composition comprising AAV particles harvested by the method of embodiment 105.
110. The method of any one of embodiments 27-54, further comprising culturing the eukaryotic cells in the medium prior to the transfecting step.
111. A method of increasing AAV production in a stable producer line, comprising: contacting the eukaryotic cells with a small molecule enhancer and one or more essential AAV production components, wherein the one or more essential AAV production components have constitutive and/or conditional expression through alteration of a cellular genome, and wherein the small molecule enhancer is one or more selected from the group consisting of: Compound A, Compound B, Compound C, Compound D, Compound E, Compound F, Compound G, sodium valproate, suberoylanilide hydroxamic acid (SAHA), vinblastine or vinblastine sulfate, or a combination thereof, or a salt thereof.
112. The method of embodiment 111, wherein the eukaryotic cells are transfected with DNA to enable the production of adeno-associated virus (AAV).
113. The method of embodiment 111, wherein the eukaryotic cells are not transiently transfected with DNA, and wherein the one or more essential AAV production components are stably integrated into the cellular genome.
114. The method of embodiment 111, wherein the one or more essential AAV production components comprise a first essential AAV production component and a second essential AAV production component different from the first essential AAV production component, wherein the first essential AAV production component is transiently transfected with DNA, and wherein the second essential AAV production component is stably integrated into the cellular genome.
115. The method of any one of embodiments of 111-114, wherein the eukaryotic cells are human embryonic kidney (HEK) 293 cells, or a variant thereof.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

Although the foregoing disclosure has been described in some detail by way of illustration and example for purposes of clarity of understanding, it will be obvious that certain changes and modifications may be practiced within the scope of the appended embodiments.

The following examples are offered by way of illustration and not by way of limitation.

### EXAMPLES

Embodiments of the enhancers disclosed herein and their methods of use are further defined in the following non-limiting Examples. It should be understood that these Examples, while indicating one or more preferred embodiments, are given by way of illustration only and are non-limiting. From the above discussion and these Examples, one skilled in the art can ascertain the essential characteristics of the disclosed compositions, and without departing from the spirit and scope thereof, can make various changes and modifications of the embodiments disclosed herein to adapt it to various usages and conditions. Thus, various modifications of the embodiments, in addition to those shown and described herein, will be apparent to those skilled in the art from the foregoing description. Such modifications are also intended to fall within the scope of the appended claims.

### Example 1: Selection and Titration of Candidate Small Molecules

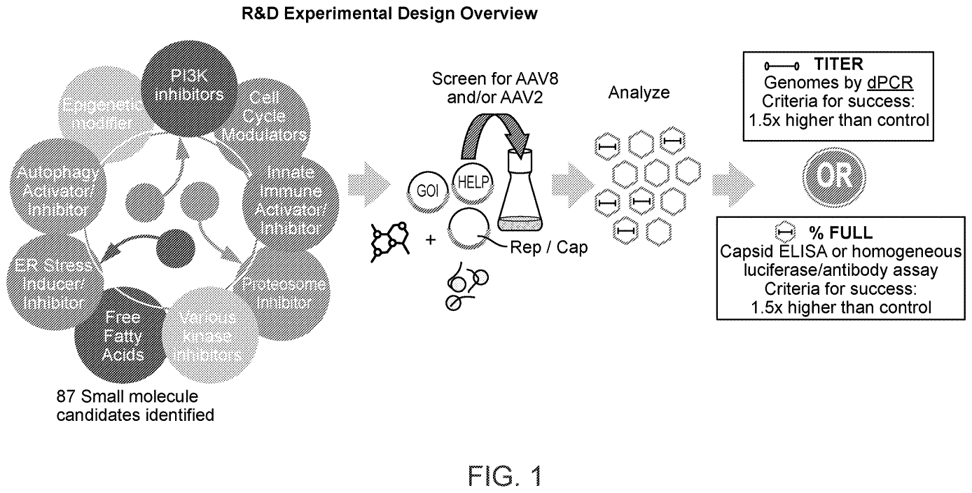

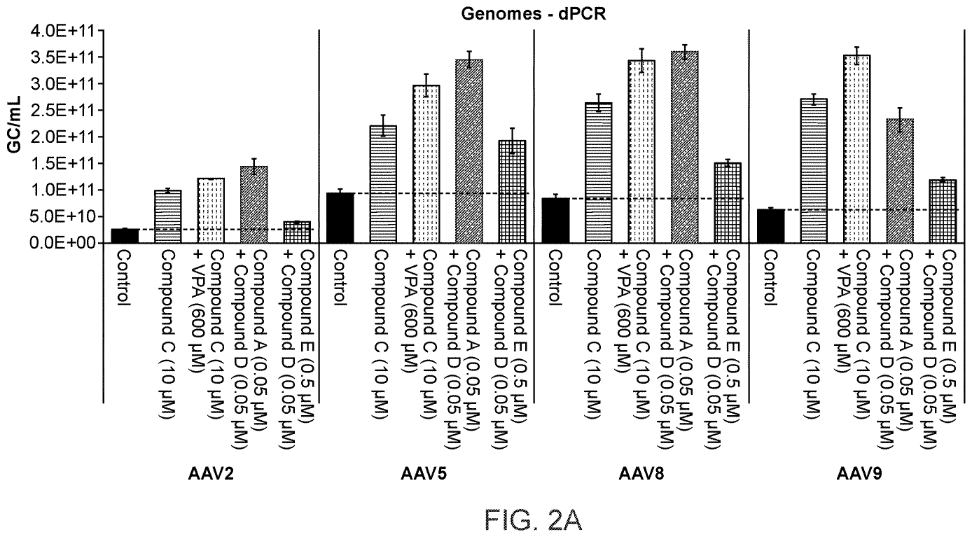

Candidate small molecules were selected via literature-based searches and encompassed general classes of epigenetic modifiers, PI3K inhibitors, cell cycle modulators, innate immune activators/inhibitors, proteosome inhibitors, kinase inhibitors, free fatty acids, endoplasmic reticulum stress inducers/inhibitors, and autophagy activators/inhibitors. Various candidate small molecule were analyzed in AAV2 or AAV8 production. The initial test concentrations of each small molecule were determined by previous uses of the molecules found in the literature for a variety of applications. For each candidate small molecule, cells were triple transfected with three plasmids encoding the rep/cap genes, gene of interest flanked by internal terminal repeats (ITR) and adenovirus helper molecules. The rep/cap plasmid encoded either AAV2 or AAV8 capsids. The plasmid DNA (pDNA) was combined with the TransIT-VirusGEN® transfection reagent and added to 293-derived suspension cells. The cells were then immediately treated with the small molecule. AAV production occurred over the course of 72 hours, after which virus was harvested. Genomes were assessed by digital PCR (dPCR), and capsids by a specific ELISA or homogeneous based luciferase assay utilizing capsid specific antibodies. The percentage of full capsids was calculated by dividing the total number of genomes by the total number of capsids. A small molecule was considered a successful enhancer of AAV production if it increased AAV as measured by viral genomes 1.5-fold over an untreated control and was not detrimental to the percentage of full capsids, or if it increased the percentage of full capsids 1.5-fold over an untreated control and was not detrimental to the total number of produced AAV as measured by viral genomes. A schematic of the experimental design is shown in FIG. 1. The experimental design may identify small molecules that enhance AAV production.

### Cell Culture

Viral Production Cells 2.0 (293-VP 2.0; Thermo Fisher Cat. No. A49784) were maintained in Viral Production Media (VPM; Thermo Fisher Cat. No. A4817901) supplemented with 4 mM GlutaMAX™ (Thermo Fisher Cat. No. 35050061). Expi293F™ Cells (Thermo Fisher Cat. No. A14527) were maintained in Expi293™ Expression Medium (Thermo Fisher Cat. No. A1435101). Both cell lines were maintained at the supplier recommended 8% CO₂ and 37° C. on a 2.5 cm orbital shaker at 125 rpm.

### Transfection and Small Molecule Addition

The day before transfection, cells were passaged to ensure the cells were actively dividing and would reach a density of 4.0-5.0×10⁶ cells/mL the day of transfection. The day of transfection, cells were seeded in 6-well non-tissue culture treated plates (Corning, Cat. No. 351146) at a density of 3.0×10⁶ cells/mL. A 1 mg/mL DNA solution composed of a 1:1:1 plasmid concentration ratio of a transfer vector (Agilent, Cat. No. 240074-51), an AAV2 RepCap Vector (Aldevron, Cat. No. pALD-AAV2) or an AAV5 RepCap Vector (Aldevron, Cat. No. pALD-AAV5) or an AAV8 RepCap vector (Genemedi, Cat. No. P-RC09) or an AAV9 RepCap vector (Genemedi, Cat. No. p-RC13), and a helper vector (Agilent Cat. No. 240071-54) was formed in water. For each milliliter of culture, 3 μL of TransIT-VirusGEN® (Mirus Bio LLC, Cat. No. MIR 6700) and 2 μg of DNA were added to a final volume of 100 μL Phosphate-buffered saline (PBS; Corning, Cat. No. 21-040-CV) for a final transfection reagent to DNA ratio of 1.5:1. This mixture was incubated at room temperature for 30 minutes to allow transfection complexes to form, after which 100 μL was added per milliliter of culture for a final complexation volume of 10%. In some instances, cells were transfected with 1 μL FectoVIR®-AAV (Polyplus, Cat. No. 101000044) per million cells and 1 μg DNA per million cells for a final transfection reagent to DNA ratio of 1:1, and a final complexation volume of 5%. In some instances, cells were transfected with 1 μL PEIPro® (Polyplus, Cat. No. 101000017) per million cells and 1 μg DNA per million cells for a final transfection reagent to DNA ratio of 1:1, and a final complexation volume of 10%. Small molecules were then added either immediately (i.e., within 5 minutes) or at the specified timepoint directly to the culture at the specified concentration. Cells were transfected for 48-72 hours before harvesting virus.

### Harvest

To harvest AAV, cells were incubated with 0.1× volume of a 10× Cell Lysis Buffer, composed of 500 mM Tris pH 8, 10% Tween®20, 20 mM MgCl₂, and 1000 U/mL Recombinant Dr. Nuclease (Syd Labs, Cat. No. BP4200) at 37° C. for 1.5 hours while shaking. Cells were then incubated with 0.1× volume of 5 M NaCl at 37° C. for 30 minutes while shaking. The lysate was centrifuged at 4,100×g for 10 minutes to remove cell debris, and the supernatant transferred to a clean tube and stored at −80° C.

### Genome and Capsid Quantitation

Genome quantitation was performed using digital PCR (dPCR) on a QIAcuity Digital PCR System (Qiagen, Cat. No. 911001). AAV lysates were diluted in a water-based buffer consisting of 1× GeneAmp PCR Buffer (Thermo Fisher Scientific, Cat. No. 4379878) and 0.05% Poloxamer 188 (Mirus Bio LLC, Cat. No. MIR 6230). Each reaction contained 1× QIAcuity Probe PCR Mastermix (Qiagen, Cat. No. 250102), 0.8 μM CMV Forward Primer (IDT; 5′-TTCCTACTTGGCAGTACATCTACG-3′) and CMV Reverse Primer (IDT; 5′-GTCAATGGGGTGGAGACTTGG-3′), 0.4 μM CMV Probe (IDT; 5′-/56-FAM/TGAGTCAAA/ZEN/CCGCTATCCACGCCCA/3IABKFQ/-3′), and 4.5 μL diluted AAV lysate in a final volume of 15 μL. Samples were loaded into a QIAcuity Nanoplate (Qiagen, Cat. No. 250021) and cycled using the following parameters: 95° C. for 10 minutes, 40 cycles of 95° C. for 15 seconds and 60° C. for 30 seconds.

Capsids were quantified using an ELISA kit for the appropriate serotypes (Progen, Cat. No. PRATV, PRAAV5, PRAAV8, PRAAV9) or a Lumit-based assay (Promega, Cat. No. VB2020) developed for Mirus Bio LLC by Promega Corporation.

### Results

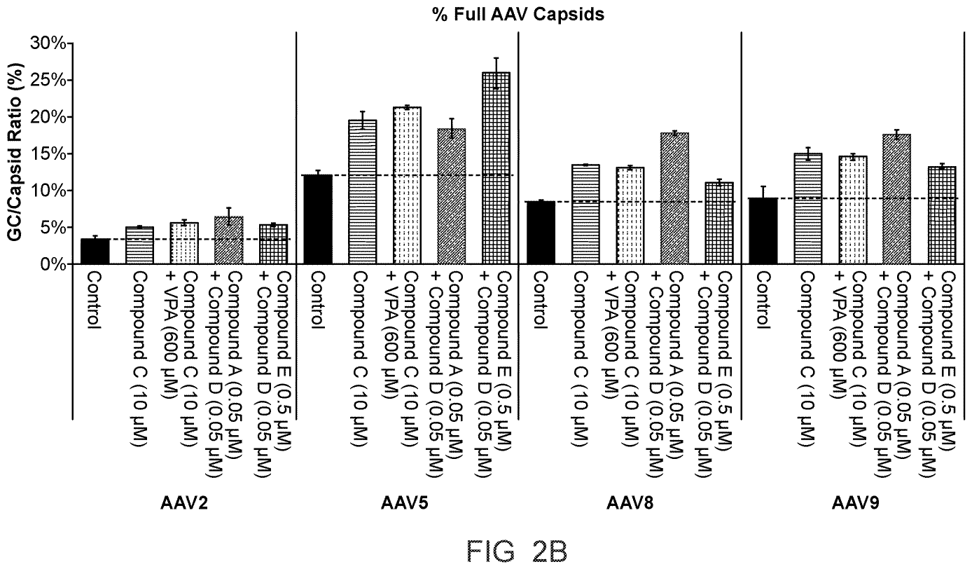

Successful enhancers of AAV8 production included Compound A (from about 0.025 μM to about 1 μM), Compound B (from about 0.0673 μM to about 0.2019 μM), Compound C (from 1 μM to about 20 μM), Compound D (about 0.05 μM), (from about 0.5 M to about 1 μM), Compound F (from about 0.5 μM to about 40 μM), Compound G (from about 0.02 μM to about 0.5 μM), SAHA (from about 0.5 μM to about 1 μM), valproic acid sodium salt (VPA; from about 400 μM to about 2500 μM), and vinblastine sulfate (from about 2.5 μM to about 15 μM), and combinations thereof. The small molecules which yielded the highest increase in AAV8 titers (“Top Hits”) were Compound A+Compound D (Compound A from 0.025 μM to 1 μM+Compound D from 0.025 μM to about 1 μM), Compound A+Compound G (Compound A from 0.01 μM to about 0.25 μM+Compound G from 0.02 μM to about 0.5 μM), Compound C (from about 1 μM to about 20 μM), Compound C+VPA (Compound C from 1.0 μM to 20 μM+VPA from 400 μM to about 600 μM), and Compound F (from about 0.5 μM to about 40 μM). The Top Hits were then assessed in the production of AAV2, AAV5, AAV8, and AAV9 and successfully induced higher genome titers of 1.5-fold or more in each serotype (FIGS. 2A-2B). According to FIGS. 2A-2B, Viral Production 2.0 cells (Thermo Fisher Scientific) grown in Viral Production Medium (Thermo Fisher Scientific) were used to generate recombinant AAV2, AAV5, AAV8 and AAV9 via transient transfection using TransIT-VirusGEN® Transfection reagent (1.5:1 reagent-to-DNA ratio (vol:wt), 2 μg/ml pDNA, 30-minute complex formation, 10% complexation volume; Mirus Bio LLC), according to the manufacturer recommended protocol. AAV was produced by transfecting pAAV-hrGFP (Agilent), pAAV-Helper (Agilent) and pAAV-RepCap (AAV2 and AAV5 Aldevron, AAV8 and AAV 9 Genemedi) at a 1:1:1 plasmid concentration ratio. Cells were transfected at a cell density of 3×10⁶ cells/ml, 2 mL in an untreated 6-well plate, shaking. Enhancer small molecules were added immediately post transfection at indicated concentrations. All AAV serotypes were harvested at 72 hours post-transfection using chemical lysis. Genome titers (FIG. 2A) were determined via dPCR using primers and a probe targeting the CMV promoter. Total assembled capsids were determined using the AAV2, AAV5, AAV8, AAV9 Titration ELISA Kits (Progen), and the GC/capsid ratio (FIG. 2B) was determined by dividing the number of genome copies by total assembled capsids for each condition. The error bars represent the range of duplicate wells.

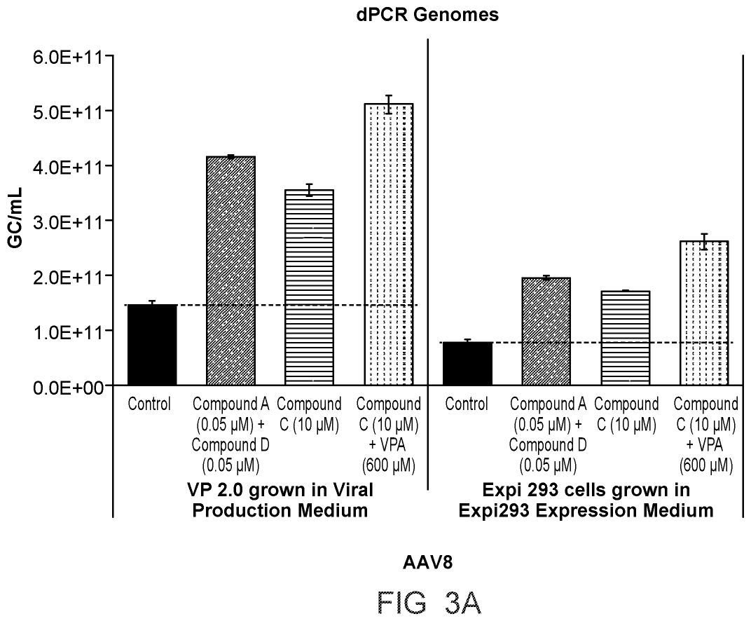

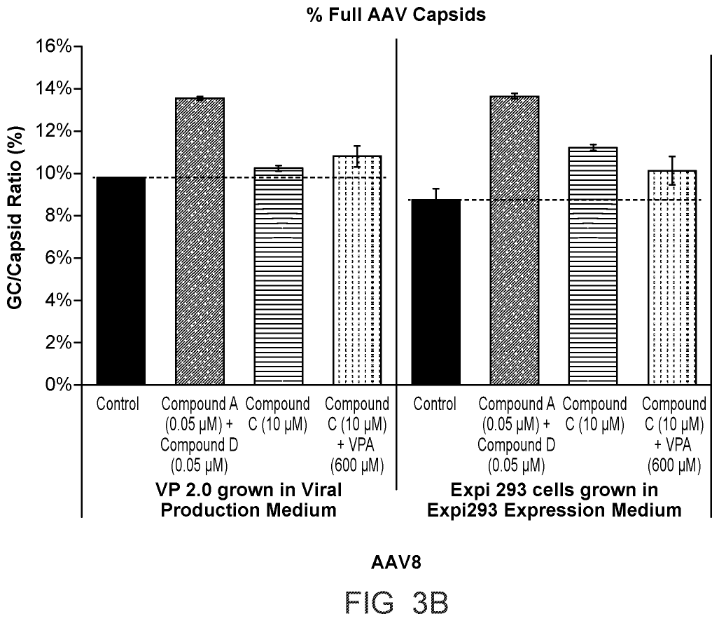

An enhancement of AAV8 production was also observed in two different suspension HEK293 cell lines: 293-VP 2.0 cells, and Expi293F™ cells (FIGS. 3A-3B). Viral Production 2.0 cells (Thermo Fisher Scientific) grown in Viral Production Medium (Thermo Fisher Scientific) and Expi293F™ cells (Thermo Fisher Scientific) grown in Expi293 Expression Medium (Thermo Fisher Scientific) were used to generate recombinant AAV8 via transient transfection using TransIT-VirusGEN® Transfection reagent (1.5:1 reagent-to-DNA ratio (vol:wt), 2 μg/ml pDNA, 30-minute complex formation, 10% complexation volume; Mirus Bio LLC), according to the manufacturer recommended protocol. AAV8 was produced by transfecting pAAV-hrGFP (Agilent), pAAV-Helper (Agilent) and pAAV-RepCap (Genemedi) at a 1:1:1 plasmid concentration ratio. Cells were transfected at a cell density of 3×10⁶ cells/ml, 2 mL in an untreated 6-well plate, shaking. Enhancer small molecules were added immediately post transfection at indicated concentrations. AAV was harvested at 72 hours post-transfection using chemical lysis. Genome titers (FIG. 3A) were determined via dPCR using primers and a probe targeting the CMV promoter. Total assembled capsids were determined using an AAV8 Custom Lumit Assay (Promega) and the GC/capsid ratio (FIG. 3B) was determined by dividing the number of genome copies by total assembled capsids for each condition. The error bars represent the range of duplicate wells.

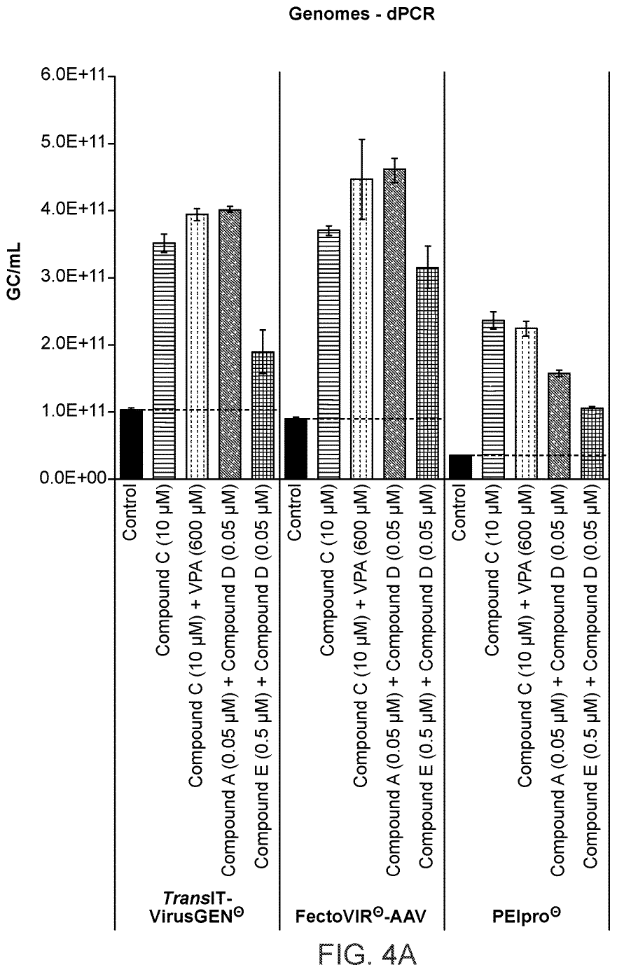

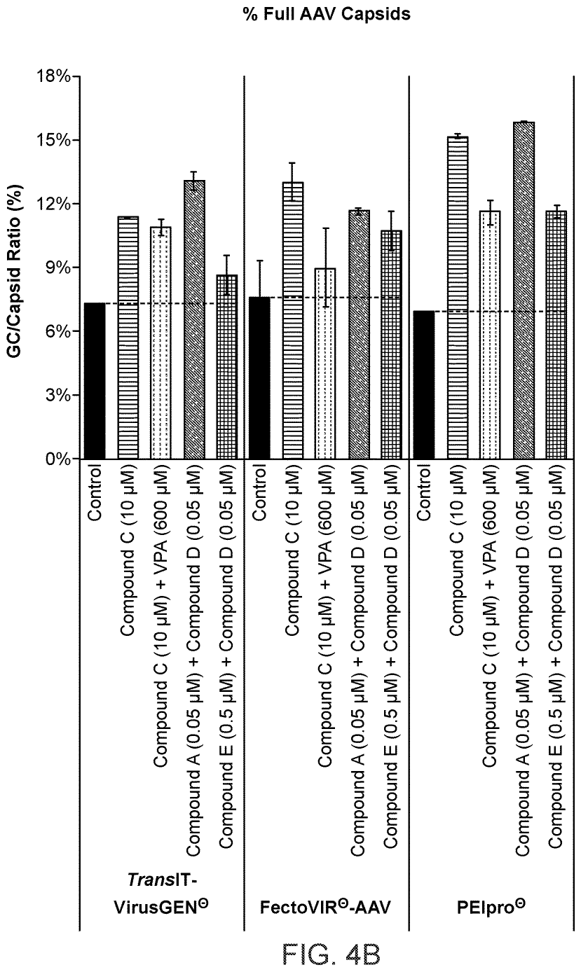

The enhancement of AAV production by transient transfection was also not restricted to TransIT-VirusGen® (Mirus Bio LLC) and could also be observed when transfecting 293-VP 2.0 cells with Fecto VIR®-AAV (Polyplus) or PEIpro® (Polyplus) (FIGS. 4A-4B). Viral Production 2.0 cells (Thermo Fisher Scientific) grown in Viral Production Medium (Thermo Fisher Scientific) were used to generate recombinant AAV8 via 1) transient transfection using TransIT-VirusGEN® Transfection reagent (1.5:1 reagent-to-DNA ratio (vol:wt), 2 μg/ml pDNA, 30-minute complex formation, 10% complexation volume; Mirus Bio LLC), 2) Fecto VIR®-AAV Transfection reagent (1:1 reagent-to-DNA ratio (vol:wt), 1 μg pDNA/1×10⁶ cells, 30-minute complex formation, 5% complexation volume; Polyplus), or 3) PEIPro® Transfection reagent (1:1 reagent-to-DNA ratio (vol:wt), 1 μg pDNA/1×10⁶ cells, 15-minute complex formation, 10% complexation volume; Polyplus) according to the manufacturer recommended protocols. AAV8 was produced by transfecting pAAV-hrGFP (Agilent), pAAV-Helper (Agilent) and pAAV-RepCap (Genemedi) at a 1:1:1 plasmid concentration ratio. Cells were transfected at a cell density of 3×10⁶ cells/ml, 2 mL in an untreated 6-well plate, shaking. Enhancer small molecules were added immediately post transfection at indicated concentrations. All AAV was harvested at 72 hours post-transfection using chemical lysis. Genome titers (FIG. 4A) were determined via dPCR using primers and a probe targeting the CMV promoter. Total assembled capsids were determined using an AAV8 Custom Lumit Assay (Promega) and the GC/capsid ratio (FIG. 4B) was determined by dividing the number of genome copies by total assembled capsids for each condition. The error bars represent the range of duplicate wells.

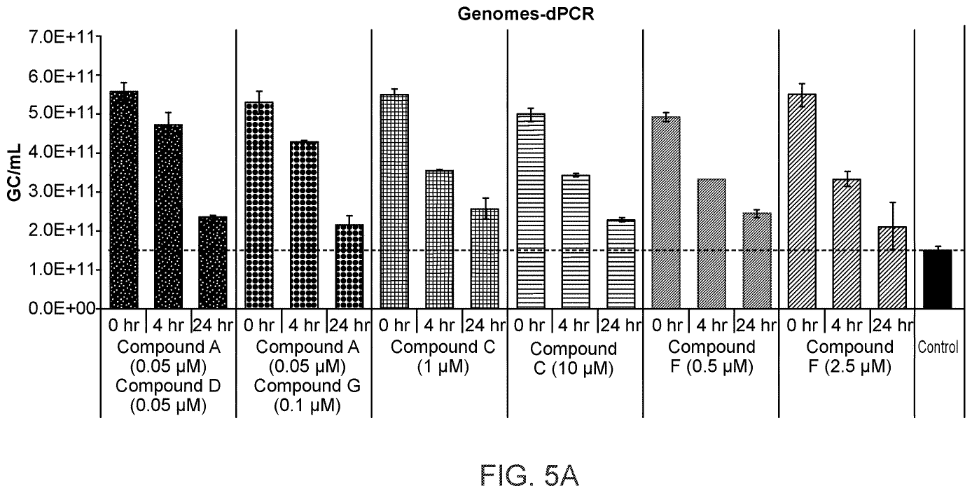

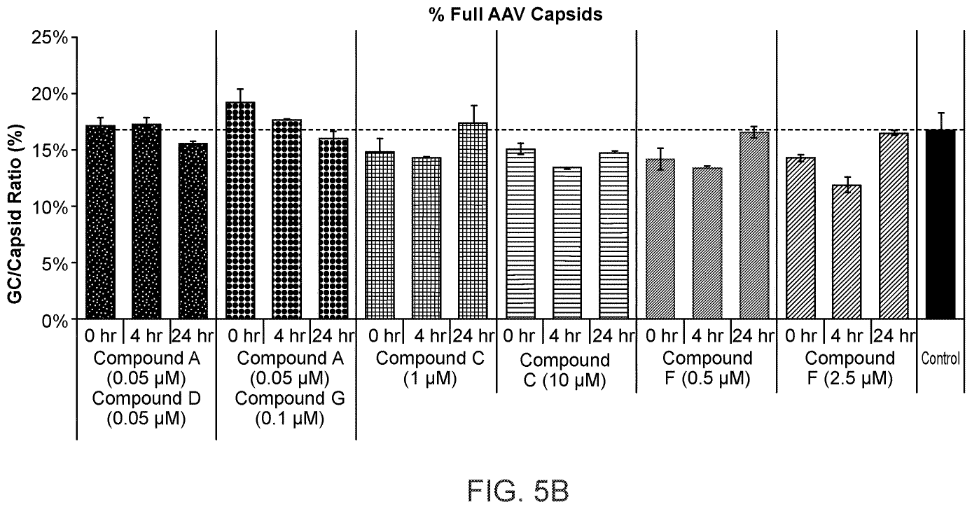

An enhancement of AAV production was observed when the small molecules were added post-transfection (FIGS. 5A-5B). According to FIGS. 5A-5B, increased AAV titers are observed when enhancers were added within 24 hours post-transfection. Viral Production 2.0 cells (Thermo Fisher Scientific) grown in Viral Production Medium (Thermo Fisher Scientific) were used to generate recombinant AAV8 via transient transfection using TransIT-VirusGEN® Transfection reagent (1.5:1 reagent-to-DNA ratio (vol:wt), 2 μg/ml pDNA, 30-minute complex formation, 10% complexation volume; Mirus Bio LLC), according to the manufacturer recommended protocol. AAV8 was produced by transfecting pAAV-hrGFP (Agilent), pAAV-Helper (Agilent) and pAAV-RepCap (Genemedi) at a 1:1:1 plasmid concentration ratio. Cells were transfected at a cell density of 3×10⁶ cells/ml, 2 mL in an untreated 6-well plate, shaking. Enhancer small molecules were added at indicated concentrations and timepoints following transfection. AAV was harvested at 72 hours post-transfection using chemical lysis. Genome titers (FIG. 5A) were determined via dPCR using primers and a probe targeting the CMV promoter. Total assembled capsids were determined using an AAV8 Custom Lumit Assay (Promega) and the GC/capsid ratio (FIG. 5B) was determined by dividing the number of genome copies by total assembled capsids for each condition. The error bars represent the range of duplicate wells.

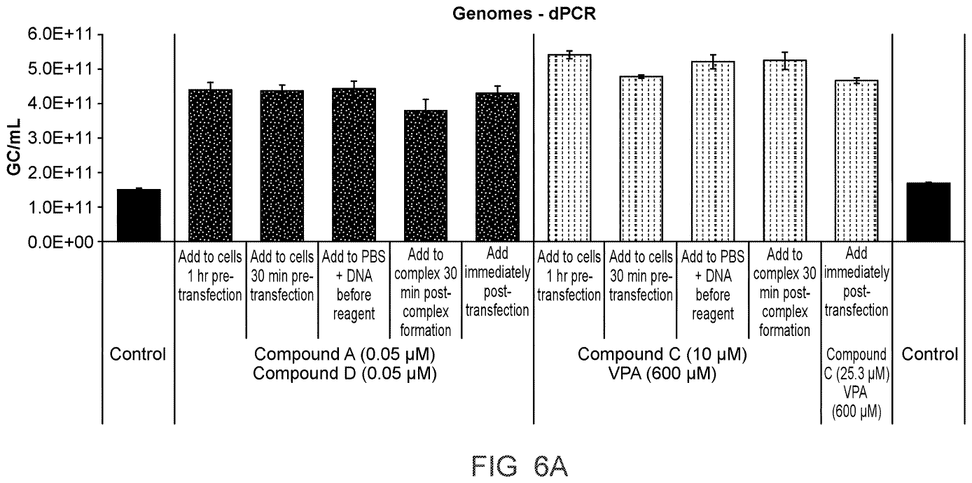

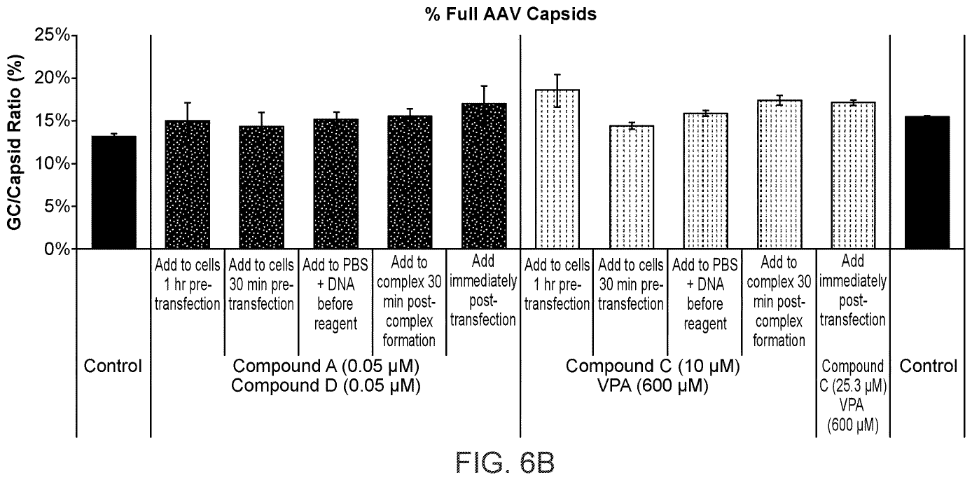

An enhancement of AAV production was observed when the small molecules were added prior to transfection (FIGS. 6A-6B). According to FIGS. 6A-6B, Viral Production 2.0 cells (Thermo Fisher Scientific) grown in Viral Production Medium (Thermo Fisher Scientific) were used to generate recombinant AAV8 via transient transfection using TransIT-VirusGEN® Transfection reagent (1.5:1 reagent-to-DNA ratio (vol:wt), 2 ug/ml pDNA, 30-minute complex formation, 10% complexation volume; Mirus Bio LLC), according to the manufacturer recommended protocol. AAV8 was produced by transfecting pAAV-hrGFP (Agilent), pAAV-Helper (Agilent) and pAAV-RepCap (Genemedi) at a 1:1:1 plasmid concentration ratio. Cells were transfected at a cell density of 3×10⁶ cells/ml, 2 mL in an untreated 6-well plate, shaking. Enhancer small molecules were added at indicated concentrations and timepoints before or following transfection. AAV was harvested at 72 hours post-transfection using chemical lysis. Genome titers (FIG. 6A) were determined via dPCR using primers and a probe targeting the CMV promoter. Total assembled capsids were determined using an AAV8 Custom Lumit Assay (Promega) and the GC/capsid ratio (FIG. 6B) was determined by dividing the number of genome copies by total assembled capsids for each condition. The error bars represent the range of duplicate wells.

Summary of the experimental results is shown in Table 1.

TABLE 1

| | | | Fold increase in |
|---|---|---|---|
| | | | genomes over no |
| Enhancer(s) | Enhancer Class(es) | Concentration Range | enhancer control |
| Compound A + | Kinase Inhibitor + Kinase | Compound A: 0.025 μM-1 μM | 1.22-6.98 |
| Compound D | Inhibitor | Compound D: 0.25 μM-1 μM | |
| Compound C | Anti-mitotic agent | 1 μM-20 μM | 1.62-6.87 |
| | (microtubule disassembly | | |
| | inhibitor) | | |
| Compound C + Valproic | Anti-mitotic agent | Compound C: 1 μM-20 μM | 2.35-5.00 |
| Acid Sodium Salt | (microtubule disassembly | Valproic Acid Sodium | |
| | inhibitor) + HDAC | Salt: 400 μM-600 μM | |
| | Inhibitor | | |
| Compound A + | Kinase Inhibitor + Kinase | Compound A: 0.01 μM-0.25 μM | 1.26-4.94 |
| Compound G | Inhibitor | Compound G: 0.02 μM-0.5 μM | |
| Compound F | Anti-mitotic agent | 0.5 μM-40 μM | 1.5-3.66 |
| | (microtubule assembly | | |
| | inhibitor) | | |
| Valproic Acid Sodium | HDAC Inhibitor | 400 μM-2500 μM | 1.5-2.11 |
| Salt | | | |
| Compound D + | Kinase + Inhibitor HDAC | Compound E: 0.1 μM-2.5 μM | 1.42-2.78 |
| Compound E | Inhibitor | Compound D: 0.01 μM-0.25 μM | |
| Compound D | Kinase Inhibitor | 0.05 μM | 1.57-1.91 |
| Vinblastine Sulfate | Anti-mitotic agent | 2.5 μM-15 μM | 1.59-1.89 |
| Compound E | HDAC Inhibitor | 0.5 μM-1 μM | 1.61-1.82 |
| SAHA | HDAC Inhibitor | 0.5 μM-1 μM | 1.52-1.81 |
| Compound B | Anti-mitotic agent | 0.0673 μM-0.2019 μM | 1.55-1.56 |
| Sodium Propionate | Cell Cycle Inhibitor | 5000 μM-10000 μM | 1.20-1.48 |

From the foregoing, it can be seen that the present disclosure accomplishes at least all of the stated objectives.

The inventions being thus described, it will be obvious that the same may be varied in many ways. Such variations are not to be regarded as a departure from the spirit and scope of the inventions and all such modifications are intended to be included within the scope of the following claims.

The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

1. A method for adeno-associated virus (AAV) production, the method comprising:
transfecting eukaryotic cells in a medium with one or more AAV plasmids using a transfection reagent; and
contacting the cells with an enhancer capable of increasing AAV production, wherein the enhancer comprises (R)-4-((8-cyclopentyl-7-ethyl-5-methyl-6-oxo-5,6,7,8-tetrahydropteridin-2-yl)amino)-3-methoxy-N-(1-methylpiperidin-4-yl)benzamide (Compound A), (S)-1,2,3,10-tetramethoxy-7-(methylamino)-6,7-dihydrobenzo[a]heptalen-9 (5H)-one (Compound B), [(1S,2S,3R,4S,7R,9S, 10S, 12R, 15S)-4-acetyloxy-1,9,12-trihydroxy-15-[(2R,3S)-2-hydroxy-3-[(2-methylpropan-2-yl)oxycarbonylamino]-3-phenylpropanoyl]oxy-10,14,17,17-tetramethyl-11-oxo-6-oxatetracyclo[11.3.1.0^{3,10}.0^{4,7}]heptadec-13-en-2-yl]benzoate (Compound C), 3-(4-(4-(2-(3-((dimethylamino)methyl)phenyl)-1H-pyrrolo[2,3-b]pyridin-4-yl)-1-ethyl-1H-pyrazol-3-yl)phenyl)-1, 1-dimethylurea (Compound D), 4-(dimethylamino)-N-(7-(hydroxyamino)-7-oxoheptyl)benzamide (Compound E), [(1S,2S,3R,4S,7R,9S, 10S, 12R, 15S)-4, 12-diacetyloxy-15-[(2R,3S)-3-benzamido-2-hydroxy-3-phenylpropanoyl]oxy-1,9-dihydroxy-10, 14, 17, 17-tetramethyl-11-oxo-6-oxatetracyclo[11.3.1.0^{3.10}.0^{4,7}]heptadec-13-en-2-yl]benzoate, (Compound F), N-[2-[(1R,8S)-4-[[4-(cyclobutylamino)-5-(trifluoromethyl)pyrimidin-2-yl]amino]-11-azatricyclo[6.2.1.0^{2,7}]undeca-2 (7), 3,5-trien-11-yl]-2-oxoethyl]acetamide (Compound G), sodium valproate, suberoylanilide hydroxamic acid (SAHA), or vinblastine sulfate, or a combination thereof.

2. (canceled)

3. The method of claim 1, wherein the enhancer comprises Compound A and Compound D.

4. The method of claim 3, wherein Compound A is at a concentration from about 0.025 μM to about 1.0 μM, and wherein Compound D is at a concentration from about 0.025 μM to about 1.0 μM.

5-25. (canceled)

26. The method of claim 1, wherein the enhancer increases viral genome titer of the culture and/or the percentage of full capsids at least 1.5-fold, at least 2-fold, at least 3-fold, at least 4-fold, at least 5-fold, or at least 6-fold.

27-54. (canceled)

55. A composition comprising:
an enhancer capable of increasing adeno-associated virus (AAV) production, wherein the enhancer comprises (R)-4-((8-cyclopentyl-7-ethyl-5-methyl-6-oxo-5,6,7,8-tetrahydropteridin-2-yl)amino)-3-methoxy-N-(1-methylpiperidin-4-yl)benzamide (Compound A), (S)-1,2,3,10-tetramethoxy-7-(methylamino)-6,7-dihydrobenzo[a]heptalen-9 (5H)-one (Compound B), [(1S,2S,3R,4S,7R,9S, 10S, 12R, 15S)-4-acetyloxy-1,9,12-trihydroxy-15-[(2R,3S)-2-hydroxy-3-[(2-methylpropan-2-yl)oxycarbonylamino]-3-phenylpropanoyl]oxy-10,14, 17,17-tetramethyl-11-oxo-6-oxatetracyclo[11.3.1.0^{3,10}.0^{4,7}]heptadec-13-en-2-yi]benzoate (Compound C), 3-(4-(4-(2-(3-((dimethylamino)methyl)phenyl)-1H-pyrrolo[2,3-b]pyridin-4-yl)-1-ethyl-1H-pyrazol-3-yl)phenyl)-1, 1-dimethylurea (Compound D), 4-(dimethylamino)-N-(7-(hydroxyamino)-7-oxoheptyl)benzamide (Compound E), [(1S,2S,3R,4S,7R,9S, 10S, 12R, 15S)-4, 12-diacetyloxy-15-[(2R,3S)-3-benzamido-2-hydroxy-3-phenylpropanoyl]oxy-1,9-dihydroxy-10, 14, 17, 17-tetramethyl-11-oxo-6-oxatetracyclo[11.3.1.0^{3,10}.0^{4,7}]heptadec-13-en-2-yl]benzoate, (Compound F), N-[2-[(1R,8S)-4-[[4-(cyclobutylamino)-5-(trifluoromethyl)pyrimidin-2-yl]amino]-11-azatricyclo[6.2.1.0^{2,7}]undeca-2 (7), 3,5-trien-11-yl]-2-oxoethyl]acetamide (Compound G), sodium valproate, suberoylanilide hydroxamic acid (SAHA), or vinblastine sulfate, or a combination thereof.

56. (canceled)

57. The composition of claim 55, wherein the enhancer comprises Compound A and Compound D.

58-86. (canceled)

87. A method for producing recombinant adeno-associated virus (AAV), comprising:
culturing eukaryotic cells in a medium;
transfecting the eukaryotic cells in the medium with one or more AAV plasmids using a transfection reagent; and
adding to the medium one or more enhancers increasing AAV production, wherein the one or more enhancers comprise a first enhancer and a second enhancer, wherein the first enhancer and the second enhancer are synergistic to increase the AAV production.

88. The method of claim 87, wherein each of the one or more enhancers is independently (R)-4-((8-cyclopentyl-7-ethyl-5-methyl-6-oxo-5,6,7,8-tetrahydropteridin-2-yl)amino)-3-methoxy-N-(1-methylpiperidin-4-yl)benzamide (Compound A), (S)-1,2,3,10-tetramethoxy-7-(methylamino)-6,7-dihydrobenzo[a]heptalen-9 (5H)-one (Compound B), [(1S,2S,3R,4S,7R,9S, 10S, 12R, 15S)-4-acetyloxy-1,9,12-trihydroxy-15-[(2R,3S)-2-hydroxy-3-[(2-methylpropan-2-yl)oxycarbonylamino]-3-phenylpropanoyl]oxy-10,14,17,17-tetramethyl-11-oxo-6-oxatetracyclo[11.3.1.0^{3,10}.0^{4,7}]heptadec-13-en-2-yl]benzoate (Compound C), 3-(4-(4-(2-(3-((dimethylamino)methyl)phenyl)-1H-pyrrolo[2,3-b]pyridin-4-yl)-1-ethyl-1H-pyrazol-3-yl)phenyl)-1, 1-dimethylurea (Compound D), 4-(dimethylamino)-N-(7-(hydroxyamino)-7-oxoheptyl)benzamide (Compound E), [(1S,2S,3R,4S,7R,9S, 10S, 12R, 15S)-4, 12-diacetyloxy-15-[(2R,3S)-3-benzamido-2-hydroxy-3-phenylpropanoyl]oxy-1,9-dihydroxy-10, 14, 17, 17-tetramethyl-11-oxo-6-oxatetracyclo[11.3.1.0^{3,10}.0^{4,7}]heptadec-13-en-2-yl]benzoate, (Compound F), N-[2-[(1R,8S)-4-[[4-(cyclobutylamino)-5-(trifluoromethyl)pyrimidin-2-yl]amino]-11-azatricyclo[6.2.1.0^{2,7}]undeca-2 (7), 3,5-trien-11-yl]-2-oxoethyl]acetamide (Compound G), sodium valproate, suberoylanilide hydroxamic acid (SAHA), vinblastine, or vinblastine sulfate, or a salt thereof.

89. (canceled)

90. The method of claim 87, wherein each of the one or more enhancers is independently an epigenetic modifier, a P13K inhibitor, a cell cycle modulator, an innate immune activator/inhibitor, a proteosome inhibitor, a kinase inhibitor, anti-mitotic agent or microtubule disassembly inhibitor, a histone deacetylase (HDAC) inhibitor, a free fatty acid, an endoplasmic reticulum stress inducer/inhibitor, or an autophagy activator/inhibitor.

91. The method of claim 90, wherein the one or more enhancers comprise a kinase inhibitor.

92. The method of claim 90, wherein the one or more enhancers comprise an anti-mitotic agent or microtubule disassembly inhibitor.

93. The method of claim 90, wherein the one or more enhancers comprise an HDAC inhibitor.

94. The method of claim 87, wherein the eukaryotic cells are 293 cells or a derivative of 293 cells.

95-104. (canceled)

105. The method of claim 87, further comprising: culturing transfected eukaryotic cells for a period time sufficient for expression of the AAV; and harvesting the AAV.

106. The method of claim 105, wherein AAV particles harvested in claim 105 are at least 1.5-fold, at least 2.0-fold, at least 3.0-fold, at least 4-fold, at least 5-fold, or at least 6-fold as many AAV particles harvested than a culture in absence of adding the one or more enhancers.

107. The method of claim 87, wherein the transfection reagent comprises a cationic lipid, a helper lipid, a cationic polymer, or a combination thereof.

108. (canceled)

109. A composition comprising AAV particles harvested by the method of claim 105.

110-115. (canceled)
